SEQUENCE LISTING (30 sequences)

SEQ ID NO: 1 - DNA, 806 bp, Saccharum sp.
cggacgcgtgggcgcggcactactcgccccgatttccctccggctgccggcgtccgtgtcgcgggaatcgttcgtggaagcggccggatcccggcttttgatcggagagagagggagggagggagggaggggatggacccggcggcggtgtgtgtgcagatggagaagcactcgacggaggcgggcttcgtggatggcggtgtccaggacgcctgcgacgacgcctgcagcatctggctggaggccttctccgaacaggaccccttcggggtgaccagctggaggcacgaggtccaccttcagtgcatgctcgagtggtgccgaagagcttccaagtgtccatgggttggctagccattcagctgtacggccccatgagcccagagcttcctgcagcttgttagggatgagcggaacgctccagaagattactccggaaccacaaccattttgtcgccttcacttgttcgggaactttacgtcccatccgttcacgagatggtggagggggggagggggtcctcctttctttgtttctgtttcttgccattacaggatcgcctggcttgcatgacaagaggccggtcagtatggtggatggtcatgggcttggtatggccacgccccagtccgttcgtttcactgtcgtgagggatccagggggtgctattttcgtttttccttccccctcagagggggaccggtcatgtcttcagtggtcttccttctaagcgcttgctcttggggctcgcgagagaaaccagttgaaaacgcgtttgactcccacggctcatgatcggccctgt

SEQ ID NO: 2 - DNA, 947 bp, Saccharum sp.
ggactgcgacgtcgacggcgaggaccagggctgcgagggcggcctcatggacgacgcgttccagttcatccagcgccgcggcgggctggcctcggagtccgggtacccgtaccagggcgaggacgggtcgtgccgctcgtccgccgcatccgcgctggtggcatccatccggggccacgaggacgtgccgcgcaacaacgaggccgcgctggcggcggccgtggcgaaccagcccgtgtccgtggccatcaacggcgaggactacgcgttccggttctacgacagcggcgtgttgggcggggagtgcggcacggacctcaaccacgccatcaccgccgtcgggtacggcacggcggccgacggcagcaggtactggctgatgaagaactcgtggggcgcgtcgtggggcgagggcggctacgtccgcatccgccgcggcgtccgcggggagggcgtctgcggcctcgccaagctgccgtcctaccccgtctaattaagccaggcccagcggcgtgcgtgcgggccgatcgcgtccaccaatgtaaccatcgcagcatacagtacgcatgatactgttacatctatctatctatctatctatctatctatctatctatctatctatctatctatctatcgaagacacaagcgatgcacgcacggctttcgtggtcctatagctagcacgtacaccgcgcgcacggcatgtcacacgtgcatggcatatttgactagctacgaatttactacttattagattatatatcctttatatgactatgatgtgtgtagtatagtgatggtcgaaatcacgacccaagcatcatatattgtacgtactctcagccaaaagaacgtcgctgaggtattagttggaccttacgtgtgaagtgaagcccacgtgtattgtaatgtgcagattggcttgaatgaaaccagttaagtgt

SEQ ID NO: 3 - DNA, 496 bp, Saccharum sp.
ccagtcgattttggatggacgacgtgtcctggaattaggaaggtatgggtgctatgctctaactctaacggcttcttttgtttctttctttctttctttctttctttctttctttctttctttctttctttctttctttcattcctgaccaattcaatccaagcggccactttatatatacaactacccctgttattgccctgttgggctcaataagtaacttaggatatcgatatatccatctctcaggtttttcttctttgctgttagcactgacttgaggcatgtgatgtgtttttttatcatgctgtcaaaactttgcaaaaccattcctactgtgtcaacccaccagaaaattgtagtcctaagcagctggttggatgatgtgataatgattccgttgtatactggtataataaacaaaaggctatttcatttgacctgaataccctactgtaataatgattttgtggtgcagcaaacaaaatctctattt

SEQ ID NO: 4 - DNA, 559 bp, Saccharum sp.
ctactctgaagattgaagaggaatttaagcagctgatgggtacatacaggtaaggccattaacttatggctggtgcatctagtgatgctgaggcctactcatgaaggtctccatcatctatttccctccatatattggcaaaagaacagttaacttatcattgtgttcttctagtgaagcaggggcctcaagttgttcttattcttacctaaaaataccgtgttattgtccttttcttttcttttcttttcttttcttttcttttcttttcttttcttttcttttcttttcttttcttttcttttcttttcttttcttttcagtaaacctattgagccagatcgcctgtttgactgtctacccaagtctctgcggccaacaaaaggtgaccatgagaccgatggaggtggccgttctgatcatccatccaaaggcttggagactgccgtatacaggaccccaacactaattcctcccagaatattgcctctcatgaatgacatagctcagcagttggttcaggctggaaatcagcaaacttgctacaaaatctacagag

SEQ ID NO: 5 - DNA, 852 bp, Saccharum sp.
ctgctgctgtacattctgttgcctttgcaattgatggtgatatgtcaacaggttcttagattacatggatttttcgtgggaactaaactactccaaaactatcagttgttcattctttactatgtacacatctggttcaacaggcctacaaagagagcagtatacattattattcttaagaagaagaggtaatgatgaatccatccaaaggtatggtaccttctaaaccgaattaatcggaaataccaagaactacaaacagcagccaccgtgagacatttttccattcagactcgtcctattacttgcatctgcattccttgcttttttacaactctttgacaatgcacttctcacaatctgcaccctgccctctccagacctcagctgcatgtgttgtctgtcagcctaggaaaataatggaaactgctagtatcagtcagtttgctagctctccctctccctctccctctccctctccggggtgatccctgccatgcggaggaaggttcatctccatccatgctccagctgctgaagatgtcggtatgtttctaagactgaacaatttcaattgaatccagataaatccatatcatctactcctctgaactgatttgcagaggcacccggggatcggatttgagtagaagtattcctcctgccttggacttgcaccacgcaatctctcatacggacctccgagtctattctcatggtgcttcctcagcacaatctggaactcactccaggacatagaaccttcatccagcttgtcaataagctccacaaacttcagcctgtcagggtttatggtttttctgaacccttcaaatctcctgtgcccttgga

SEQ ID NO: 6 - DNA, 408 bp, Saccharum sp.
catagtaaatctggaacaaaatagaatatcacacccacatggaccgaatcatattcctcgagtatcaccatacacaaatagttagggtataaaacaacaaaagaaggaatgtacaattgcaacgcaaaccgccatccatcggtacacagccttcataggctatctactgacgaggccaccatagaacatgtatgtatcaatgtatacgttcgtgtgtgtatatatactattatattaggaaaaccaaaagctcttccaaacacatcaccaaaagtatatactgtgctgtgctgtgctgtgctgtgctgtgctgtgctgtgctgtgctgcgccgccattcctctcctcccaattgccgctgatccatgctcacagcacaatcaccttgtgcttgctgcggaatggtgac

SEQ ID NO: 7 - DNA, 1343 bp, Saccharum sp.
cgcgtccgaccacacgcagaccgtggccggttgggccgtcatgaacgagtctggcaaggtcgagcccttcgtcttcaagagaagggagaacggcgtggacgacgtgacgatcaaggtgcagtactgcggcatgtgccacaccgacctgcacttcatccagaacgactggggcatcaccatgtacccgctggtcccggggcacgagatcaccggcgtggtgaccagggtggggagcaacgtctccggcttccgcgccggcgaccgcgtcggcgtgggctgcatcgccgccgcctgcctcgactgcgaccactgccgccggtccgaggagaactactgcgacaaggtgacgctcacctacaacggcgtcttctgggacggcagcgtcacgtacggcggctactcccggatgctggtggcgcacaagcggttcgtggtgcgcgtcccggacacgctcccgctggacgccgcggcgccgctgctgtgcgcggggatcaccgtgtacagccccatgaagcaccacggcatgctgcggtcacccggcggcagcctgggcgtggtggggctgggaggactggggcacgtcgccgtcaagttcgccaaggcgttcggcctccgcgtcaccgtcatcagcacgtcgccggccaaggagagggaggcgagggagcggctcggcgccgaccatttcgtcgtcagcaccgaccagaagcagatgcaggccatggcgagaagcctcgactacgtcatcgacaccgtctcggccaagcactcgctgggccccatcctggagctgctcaaggtgaacggcaagctcgtgctcgtcgccgcgccggaccagcccgtcgagctcccgtcgttcccgctcatctttgggaagaggacggtgagcgggagcatgacgggaggaatgaaggagacgcaggagatgctggacctgtgcggccagcacaacatcacctgcgacatcgagctcgtctccacggacgggatcaacgaggcgctcgcgcgcctcgcgcgcaacgacgtccgctaccgcttcgtcatcgacatcgccggcgactcaaactcaaagctgtagttcaattaattcgttgcattatatcaagtctcaacaatcaacattgccggcactgctggtggttgattgctatatctatatctatatctatatctatatctatatctatatctatatctatatctatatctatatctatgtctatatctatagtgtgtgtcagctttagcaatatgatcagtggtgatcacttgagccacgagagtgtatgtaatggttgatgtactagctgcctcttgatgtgggacagtttggacgactttaattt

SEQ ID NO: 8 - DNA, 465 bp, Saccharum sp.
acaaaaagaaacacctttgtatatatatttgtagtacacaataatctcgtatgtaccaatgtcttctgttcctctgaccaagcaggagcagagtatacagaaaaagagtaaaaaaaaaagagtgtgtatcatgggctagaacaaaattaacatattggttactacgtacaatccctaatcttccaaatgcgtcttcttcttcttcctcttgtctgtaaaaacgtaggggaatgggtggataattcagggaggaaaggcaggccaagatgaatgaagggccaaaaagaaaaagaaaaagaaaaagaaaaaggatcagaattcagaagtattccaagttgaagtcgaaatgcgccaggctctgctcctggccgcagggcagcaagaacttgtgatccagcgcgtcgtcgtcgtcgtccaccacctgccccccgccgctgccgtaccctccgctgtgctgctgctgct

SEQ ID NO: 9 - DNA, 913 bp, Saccharum sp.
gtctccgatcgcgaccccgatcgaaatcccgtcaagcttcagcggggcatccatccccgtcgtaccaacccaccagtgttcttcctcccttcctcccttccctgtataaaaacgccaccaccgatcgtggtttcgtgctctcgcttcgctcccttaatttatcatcactcctaatttattttatttcttattatcatatgtattgatttcgctcgccctcctttcccgactgttcccgtgcctcacaccggctcggattgcttgctctcgcttgcttgtgtcagaataagattccgcgcaagtttggattcaccgaatccaccgatctctcgcgccccgcggtcgcgcgcggtggaatttcggcctgggcggctgggcgccgccgattcgggcctctccggaagggtgaccccgggtgtggttcgcctaatctccgcccaaatttcagctgccttagtgtggaactcgtcgtgattttcgactgtgcgcttggctttcctgtcctgtcctgtcctgtcctgtcctgtcccgtgttgtggcttcagcatttcaggggatggggcgccgcgggttgctcacggccgcgcttctgctgtgcttccttgctgtttgctccggcagagaacttacgatcaaacagaacccttccactaccatatacaactcgactcttgcccagatacttgtggagtatgctgctgcaatctatactgctgatttgacagaattgtttacttggacatgtgccagatgtggtgacttgatcaaggggttcgagatgatagagatcattgtagatgtggagaactgcttgcaggcatatgttggttttgccagcgatataaacgccgtcgtcgttgtttttagaggaactcaagagaacagcatccagaattggatagaagacttgcta

SEQ ID NO: 10 - DNA, 737 bp, Saccharum sp.
ttgggtttgaactacaaccttgtgggaggctccaacacaaaacagagcatactcgaggccatggacgtaaaagtgtagctaccaagtctactgctcgaactaaccagaaagatggcggcggcggcggcggctgggcggcgggttctccggcgacgttccgatcttccacgcggaaaacctcgtcagcaacgtcaaatcaatcaactacagccggacattcttgtcaatcattagtggagttgttgctggaatatgggggtttacaggcttgatgggatttgtgttctactttcttgtgatgatggttgcatctctgatgcttttggtgaaggccaaattttcggtccatacatactttgacagttggaacaggattataattgaaggagttcttgggggcctgatgtctttcgtgttgttttggacatttgcctatgacattgtccacatcttctgatggatgaaacaagagctctctgaagaactgaggaaccatgtaatccctccctgtatgagacggaattattgtgtaattgcacctactccgccatctggggactatgttcagtggactaccgtcctcaattagtctcttgctgtatactagtcagaagcctgaacttcagaaagttacaagactataaatgaactcaggttagcagactgtcagacttgtgtaaccccgcagctgtctggaagcgtggtgctcggttctgcctgctaaatggacatgcagt

SEQ ID NO: 11 - DNA, 19 bp, Artificial Sequence
  Description of Artificial Sequence Synthetic oligonucleotide
cactactcgccccgatttc

SEQ ID NO: 12 - DNA, 19 bp, Artificial Sequence
  Description of Artificial Sequence Synthetic oligonucleotide
cgagtgcttctccatctgc

SEQ ID NO: 13 - DNA, 20 bp, Artificial Sequence
  Description of Artificial Sequence Synthetic oligonucleotide
tcgcgtccaccaatgtaacc

SEQ ID NO: 14 - DNA, 20 bp, Artificial Sequence
  Description of Artificial Sequence Synthetic oligonucleotide
gcgtgcatcgcttgtgtctt

SEQ ID NO: 15 - DNA, 20 bp, Artificial Sequence
  Description of Artificial Sequence Synthetic oligonucleotide
ggatggacgacgtgtcctgg

SEQ ID NO: 16 - DNA, 22 bp, Artificial Sequence
  Description of Artificial Sequence Synthetic oligonucleotide
ataaagtggccgcttggattga

SEQ ID NO: 17 - DNA, 20 bp, Artificial Sequence
  Description of Artificial Sequence Synthetic oligonucleotide
gaagcaggggcctcaagttg

SEQ ID NO: 18 - DNA, 21 bp, Artificial Sequence
  Description of Artificial Sequence Synthetic oligonucleotide
gtcaaacaggcgatctggctc

SEQ ID NO: 19 - DNA, 20 bp, Artificial Sequence
  Description of Artificial Sequence Synthetic oligonucleotide
aatctgcaccctgccctctc

SEQ ID NO: 20 - DNA, 20 bp, Artificial Sequence
  Description of Artificial Sequence Synthetic oligonucleotide
cagctggagcatggatggag

SEQ ID NO: 21 - DNA, 21 bp, Artificial Sequence
  Description of Artificial Sequence Synthetic oligonucleotide
acgaggccaccatagaacatg

SEQ ID NO: 22 - DNA, 21 bp, Artificial Sequence
  Description of Artificial Sequence Synthetic oligonucleotide
gcaattgggaggagaggaatg

SEQ ID NO: 23 - DNA, 21 bp, Artificial Sequence
  Description of Artificial Sequence Synthetic oligonucleotide
ggcactgctggtggttgattg

SEQ ID NO: 24 - DNA, 21 bp, Artificial Sequence
  Description of Artificial Sequence Synthetic oligonucleotide
tcccacatcaagaggcagcta

SEQ ID NO: 25 - DNA, 21 bp, Artificial Sequence
  Description of Artificial Sequence Synthetic oligonucleotide
ggcaggccaagatgaatgaag

SEQ ID NO: 26 - DNA, 19 bp, Artificial Sequence
  Description of Artificial Sequence Synthetic oligonucleotide
agcacagcggagggtacgg

SEQ ID NO: 27 - DNA, 21 bp, Artificial Sequence
  Description of Artificial Sequence Synthetic oligonucleotide
gattttcgactgtgcgcttgg

SEQ ID NO: 28 - DNA, 21 bp, Artificial Sequence
  Description of Artificial Sequence Synthetic oligonucleotide
aagttctctgccggagcaaac

SEQ ID NO: 29 - DNA, 21 bp, Artificial Sequence
  Description of Artificial Sequence Synthetic oligonucleotide
aaacagagcatactcgaggcc

SEQ ID NO: 30 - DNA, 21 bp, Artificial Sequence
  Description of Artificial Sequence Synthetic oligonucleotide
acgttgctgacgaggttttcc

(19) United States
(12) Patent Application Publication
(10) Pub. No.: US 2010/0021916 A1
(43) Pub. Date: Jan. 28, 2010

(54) MICROSATELLITE-BASED FINGERPRINTING SYSTEM FOR SACCHARUM COMPLEX

(75) Inventors: Sizuo Matsuoka, Araras (BR); Walter Maccheroni, JR., Campinas (BR)

Correspondence Address: FOLEY AND LARDNER LLP;SUITE 500, 3000 K STREET NW, WASHINGTON, DC, 20007, US

(73) Assignee: CANAVIALIS S.A., Campinas, SP (BR)

(21) Appl. No.: 12/522,612

(22) PCT Filed: Jan. 10, 2008
(86) PCT No.: PCT/BR08/00009

§ 371 Date: Sep. 14, 2009

Related U.S. Application Data
(60) Provisional application No. 60/884,705, filed on Jan. 12, 2007.

(51) Int. Cl. C12Q 1/68; C12N 15/11

(52) U.S. Cl. 435/6; 536/23.6; 536/24.33

(57) ABSTRACT

This invention relates to Sacchanum spp. microsatellites and their flanking sequences, methods for microsatellite isolation, and methods applicable to use these sequences for fingerprinting and that may be used for applications such as: identification of Saccharum complex accessions, management of genrmplasm collections, selection of progenitors, distinction between varieties, identification of hybrids, introgression of desirable genomic sequences, paternity determination, mapping of genes and quantitative trait loci (QTLs) and development of MAS (marker-assisted selection) procedures.

### RELATED APPLICATION

This application claims priority of U.S. Provisional Application Ser. No. 60/884,705 filed Jan. 12, 2007, and incorporated by reference in its entirety.

### FIELD OF THE INVENTION

This invention concerns the molecular analysis of Saccharum complex accessions. This invention relates to Saccharum spp. microsatellites and their flanking sequences, methods for microsatellite isolation, and methods applicable to use these sequences for fingerprinting and that may be used for applications such as: identification of Saccharum complex accessions, management of gemiplasm collections, selection of progenitors, distinction between varieties, identification of hybrids, introgression of desirable genomic sequences, paternity determination, mapping of genes and quantitative trait loci (QTLs) and development of MAS (marker-assisted selection) procedures.

### BACKGROUND OF THE INVETION

The search for renewable energy resources places sugarcane in an eminent position in the world crop scenario. The juice extracted from the succulent stalks of this perennial grass responds for 75% and more than 30% of the global sugar and ethanol production, respectively (Ming et al., 2006, Plant Breed. Rev. 27: 15-118; Orellana and Bonalume Neto, 2006, Nat. Biotechnol. 24: 232).

With the increasing global demand for ethanol, sugarcane cultivation is expanding to new areas and there is a constant demand for higher productivity in the fields (Thomas and Kwong, 2001, Energy Policy 29: 1133-1143; Geller et al., 2004, Energy Policy 32: 1437-1450; Gantz, 2005, Renewable Fuel News 17(27): 1-6). New varieties adapted to new environments and improved cultivation techniques are required as much as improved varieties to increase productivity of sugar and biomass. Genetic improvement of sugarcane and release of varieties for extensive cultivation have been conduced by several breeding programs around the world (Tew, 1987, New Varieties In: Heinz D J (ed.) Sugarcane Improvement through Breeding, Elsevier Press, Amsterdam: 559-592; Machado, 2001, Sugarcane Variety Notes “An Internacional Directory” 7^{th} edition). Overall, the results obtained by conventional breeding for more than a century now, based mainly on germplasm exchange and development of breeding techniques, has permitted the crop to be grown widely in tropical and sub-tropical areas and has produced over the years continuous increase in cane and sugar yields (Hogarth et al., 1977, Sugarcane Improvement: past achievements and future prospects In: Manjit SK (ed) Crop Improvement for the 21^{st} Century, Louisiana State University, Louisiana: 29-55; Miocque and Machado, 1977, Sugar J. 40: 9-13; Heinz and Tew, 1987, Hybridization procedures In: Heinz D J (ed.) Sugarcane Improvement through Breeding, Elsevier Press, Amsterdam: 313-342; Matsuoka et al., 1999, Melhoramento de cana-de-acúcar In: Borém A (ed.) Melhoramento de espécies cultivadas, Imprensa Universitária, Vicosa: 205-251; Berding et al., 2000, Advances in breeding technologyfor sugarcane In: Keating B A and Wilson J (eds.) Intensive Sugarcane Production: Meeting the Chalenges beyond 2000, CABI Publishing, UK: 141-156; Edmé et al., 2005, Crop. Sci. 45: 92-97; Jackson, 2005, Field Crops Res. 92: 277-290).

Sugarcane belongs to the genus Saccharum, tribe Andropogoneae, family Poaceae, with six species S. officinarum, S. robustum, S. barberi, S. sinense, S. spontaneum, and S. edule, being recognized. S. barberi and S. sinense are thought to be natural hybrids between S. officinarum and S. spontaneum whereas S. officinarum would be originated from wild populations of S. robustum at its center of origin Papua, New Guinea. Other genera such as Erianthus, Sclerostachya, Narenga, and Miscanthus are phylogenetically related to sugarcane and together they form the “Saccharum Complex” (Daniels and Roach, 1987, Taxonomy and evolution in sugarcane In: Heinz D J (ed.) Sugarcane Improvement through Breeding, Elsevier Press, Amsterdam: 7-84). Current varieties are hybrids created from initial interspecific crosses involving mainly four species S. officinarum, S. spontaneum, S. barberi, and S. sinensi (Irvine, 1999, Theor. Appl. Genet. 98: 186-194). The major part of the genome of a sugarcane hybrid comes from S. officinarum (80%), the noble cane rich in sugar, 10 to 15% from S. spontaneum, the rustic cane adapted to environment stresses and resistant to pests and diseases and some few are hybrid chromosomes (D'Hont et al., 1996, Mol. Gen. Genet. 250: 405-413; Price, 1957, Bot. Gaz. 118: 146-159; Roach, 1969, Proc. Int. Soc. Sugar Cane Technol. 13: 901-920). Most varieties are related to each other in a certain degree as they come from the same original crosses made back in the early 1900 in Java (Indonesia) and India. The genetic basis of hybrids, quantified from known pedigrees, is restricted to a small population of S. officinarum clones and few from the other specie (Arcenaux, 1967, Proc. Int. Soc. Sugarcane Technol. 12: 844-854; Tew, 1987, New Varieties In: Heinz D J (ed.) Sugarcane Improvement through Breeding, Elsevier Press, Amsterdam: 559-592). However, due to the polyploidy nature of the genus Saccharum combined with the aneuploidy occurring in the interspecific hybrids, genetic diversity estimated by molecular methods is still high (Jannoo et al., 1999, Theor. Appl. Genet 99: 171-184). True clones of S. officinarum have a basic chromosome number of 2n=80 (x=10) and S. spontaneum has 2n=40-128 (x=8). Due to the aneuploid that occurred during the nobilisation process (repeated backcrossing to S. offcinarum) that took place in the early genetic improvement of sugarcane, the total chromosome number of hybrids may vary between 100 and 130 (Bremer, 1961, Euphytica 10: 59-78).

Breeding of a commercial variety may take 12 to 15 years to complete a cycle and introgression of new traits may require several decades (Skinner et al., 1987, Selection methods, criteria and indices In: Heinz D J (ed.) Sugarcane Improvement through Breeding, Elsevier Press, Amsterdam: 409-452). The high heterosigozity of alleles in progenitors produced by interspecific hybridization, polyploidy and aneuploidy and the quantitative inheritance nature of most important traits such as field performance, sugar accumulation, fiber content and disease and pest resistances make progeny performance highly unpredictable (Hogarth, 1987, Genetics of sugarcane In: Heinz D J (ed.) Sugarcane Improvement through Breeding, Elsevier Press, Amsterdam: 255-271; Grivet and Arruda, 2001, Curr. Opin. Plant Biol. 5: 122-127; Hoarau et al., 2002, Theor. Appl. Genet. 105: 1027-1037; Ming et al., 2006, Plant Breed. Rev. 27: 15-118; Wei et al., 2006, Theor. Appl. Genet. 114: 155-164). Large numbers of crosses involving parent testing are needed to identify good pairs and selection screening schedules involve large initial populations. To overcome these bottlenecks breeders have been. developing DNA markers which are used at several stages of variety selection, increasing the chances of finding good parents and reducing time and costs of development (Butterfield et al., 2004, S. Afr. J. Bot. 70: 167-172; Manners et al., 2004, Proceedings of the 4^{th} International Crop Science Congress; Casu et al., 2005, Field Crop Res. 92: 137-147; McIntyre et al., 2005, Mol. Breed. 16: 151-161; Moore, 2005, Int. Sugar J. 107: 27-31; Aitken et al., 2006, Theor. Appl. Genet. 112: 1306-1317).

Microsatellites, also referred to as simple sequences, simple sequences repeats (SSRs), simple repetitive DNA sequences, short tandem repeats (STRs) or simple sequences motifs (SSMs) are stretches of DNA found in coding and non-coding areas of genomes of every living organism. They are composed of number of tandemly repeated short nucleotide motifs or repeats units. Microsatellites are formed by one to six nucleotide units repeated in tandem, varying in length between genomic chromosome copies and can be detected by PCR (Polymerase Chain Reaction) and are visualized by different chromatographic meanings as a code bar system made of amplified DNA pieces differing in size. A characteristic feature of microsatellites is that they are highly polymorphic. That is, each microsatellite “locus” may have a number of “allelic” forms which vary largely according to the value of ploidy (number of chromosome copies). Polymorphic STR loci are extremely useful markers in every organism for identification, paternity testing and genetic mapping. Polymorphism is a feature of microsatellites which contributes greatly to their usefulness in fingerprinting.

Microsatellites were initially described in humans (Litt and Luty, 1989, Am. J Hum. Genet. 44: 397-401), and subsequently in other mammalian species such as mice (Love et al., 1990, NucI. Acids Res. 18(14): 4123-4130), pigs (Johansson et al., 1992, J Hered. 83(3): 196-198) and cattle (Kemp et al., 1993, Animal Genet. 24: 363-365). In humans, microsatellite polymorphisms have been used widely for individual identification in, for example, paternity and forensic cases, and for mapping of genes correlating with genetic diseases. For example, U.S. Pat. No. 5,364,759 to Caskey et al discloses. typing assays for fingerprinting of human individuals for forensic and medical purposes, as well as techniques for identifying microsatellite sequences from DNA databases. Specific trimeric and tetrameric short tandem repeats (STRs) present in the human genome with characteristics suitable for inclusion in DNA profiling assays are also disclosed. U.S. Pat. No. 5,582,979 to Weber provides a large variety of specific sequences, isolated from human genomic DNA, which flank CA and GT dinucleotide repeats for use in forensic and paternity tests employing polymorphisms in the repeat area.

U.S. Pat. No. 5,580,728 to Perlin discloses a method and automated system for genotyping using amplified DNA sequences containing repetitive sequences showing polymorphism between DNA samples. This patent describes techniques for automated data acquisition and interpretation using short tandem repeats (STRs) and the steps required to build genetic maps based on such polymerase chain reaction (PCR)-amplified markers. U.S. Pat. No. 5,573,912 to Buard et al. describes a protocol for obtaining novel short tandem repeat regions from DNA using size-separated restriction enzyme digests, followed by hybridization with genomic DNA of the same species, and comparison of the hybridization pattern with that obtained using known probes containing variable tandem repeat regions. No specific sequences of immediate utility for genotyping are disclosed.

U.S. Pat. Nos. 5,369,004 and 5,378,602 to Polymeropoulos et al. disclose specific sequences suitable as PCR (polymerase chain reaction) primers for DNA repeat polymorphism detection in humans for medical purposes and genetic mapping. U.S. Pat. No. 5,650,277 to Navot et al. discloses a method of determining the exact number of oligonucleotide repeats within a microsatellite, wherein each repeat is two or three nucleotides long. This patent does not teach any specific primers, but requires previous determination of the repeat sequence within the microsatellite or of sequences flanking the microsatellite.

Microsatellites have been used for genome mapping of various plants, including rice, maize, soybean, barley and tomato, and are therefore becoming important tools for use in the preparation of genome maps. Zhao et al., 1996, in Applications of repetitive DNA sequences in plant genome analysis In: Paterson A H (ed.) Genome Mapping in Plants, R. G. Landes Co., New York: 111-125 disclose a review of the use of DNA repeat motifs in plant genome mapping. In addition to genetic mapping, microsatellites may be employed in physical mapping. For example, some types of repeats may show a specific distribution on the chromosomes (Schmidt and Heslop-Harrison, 1996, Proc. Natl. Acad. Sci USA 93(16): 8761-8765), so that different microsatellites may be useful in physical mapping of different areas of the genome.

Microsatellites have also been used for fingerprinting of many agricultural plants, as well as evaluating genetic diversity between plant cultivars, subspecies and so on. The main advantage of microsatellites is that they are often highly polymorphic, even within a species and cultivar. In addition, the microsatellite flanking sequences are often locus-specific thus providing a specific primer for reliably isolating that genome region. Examples of the use of microsatellites in plant identification include grapevine cultivar identification and evaluation of the genetic relatedness of cultivars (Thomas et al, 1994, Plant Mol. Biol. 25(6): 939-949); identifying individuals of wild yam for common parents in natural populations (Terauchi and Konuma, 1994, Genome 37(5): 794-801); variety identification of leaf mustard germplasm (Bhatia et al., 1995, Electrophoresis 16(9): 1750-1754); identification of chickpea varieties (Sharma et al., 1995, Electrophoresis 16(9): 1755-1761); maize cultivar germplasm genetic analysis (Taramino and Tingey, 1996, Genome 39(2): 277-287); and evaluation of within-cultivar variation of genetic diversity in rice (Olufowote et al., 1997, Genome 40(3): 370-380).

Microsatellite markers are being increasingly employed to locate specific, economically useful genes in plant genomes by linkage analysis. For example, STRs were used to map a microsatellite marker close to the rice Rf1 gene, a fertility restorer gene essential for hybrid rice production, by polymerase chain reaction (PCR) amplification and linkage analysis of microsatellite polymorphism (Akagi et al., 1996, Genome 39(6): 1205-1209). This marker could be employed not only in breeding fertility restorer and maintainer lines, but also in managing the purity of hybrid rice seeds.

Microsatellites are a class of DNA-based molecular marker as described above commonly used in plant improvement for gene and trait mapping and the subsequent development of MAS (Marker-Assisted Selection) procedures (Rakoczy-Trojanowska and Bolibok, 2004, Cell Mol Biol. Lett. 9: 221-238; Varshney et al., 2005, TIB 23: 48-55). Due to the capability of tagging and differentiate specific genomic regions shared by populations, microsatellites are used as fingerprinting systems that discriminate individuals. In plant breeding programs, microsatellites and other DNA markers have been successfully used for identification, classification, and management of germplasm collections, parent selection, variety distinction, introgression of desirable traits, and paternity determination (Lee, 1995, Adv Agron. 55: .265-344; Eagles et al., 2001, Aust. J. Agric. Res. 52: 1349-1356; Kirst et al., 2005, J. Hered. 96: 161-166), and are under examination by UPOV (Union for the Protection of New Varieties of Plant) to be a supporting discrimination tool for variety identification in relation to property's rights issues (UPOV 2005). UPOV evaluates adjusted descriptors by a standardized procedure known as distinctiveness, uniformity, and stability (DUS) test. Molecular markers that meet the requirements of a DUS test are under consideration by UPOV to be used for variety identification in relation to the enforcement of plant breeders' rights, technical verification and the consideration of essential derivation.

The verification of hybrid progeny from interspecific or intergeneric crosses in sugarcane improvement is essential before use can be made of these progeny in introgression studies, (Lee et al., 1998, Aust. J Agri. Res. 49:. 915-921). Traditional morphological methods of hybrid identification in sugarcane are unreliable because it is difficult to differentiate the genuine hybrids from selfed progeny or progeny arising from pollen contamination. Molecular markers provide a reliable method for identifying true hybrids.

Selection of parents in sugarcane generally involves the initial testing of potential candidates where small progenies or families from biparental or polycrosses are evaluated through the regular selection scheme of a particular breeding program (Heinz and Tew, 1987, Hybridization procedures In: Heinz D J (ed.) Sugarcane Improvement through Breeding, Elsevier Press, Amsterdam: 313-342). Those pair-wise combinations of male and female that produce elite individuals become proved parents. The same crosses are then repeated to produce large progenies for the selection of varieties. In polycrosses, only the mother(s) may be proved at the first stage but several male can be tested before finding the right father(s). Thus, polycrosses have in theory a higher probability of revealing proved parents whereas biparental crosses can be made routinely and in large quantities, having an advantage for revealing elite individuals. A polycrossing system where breeders could identify as fast as possible the male parents of selected clones may reduce time and costs of development of new varieties and should have a significant impact in the amount of achieved improvement (Buteler et al., 1997, Euphytica 96: 353-361).

Method for paternity is very important because in sugarcane, the breeding efficiency is very low; for instance, less than 0.0003% of the progeny from 7,000,000 seedlings evaluated from 1970 throught 1989 in the IAA/PlanalSucar breeding program in Brazil was released as commercial variety. It is virtually impossible to make all cross combinations among even the most elite parents used in breeding programs. Traditionally biparental crosses with only one female parent and only one male parent are used. Other strategy, frequently utilized is the multiparental crosses (or polycross) involving only one female parent and 8-40 male parents in open-pollination. The polycross approach has been used in sugarcane breeding to maximize the number of cross combinations that could be represented among progeny at the seedling stage of testing. The primary objection to using the polycross approach has been the rapid loss of pedigree information that occurs over generations of breeding. Microsatellite-based paternity analysis is proposed as an effective means for identifying the male parentage of progeny resulting from polycrosses. In some cases identifying the female parent is also necessary.

Sugarcane progenitors are classified as male or female before crossing according to the percentage of viable pollen. Usually males have more viable pollen than females but self-pollination is not completely avoided by natural self-incompatibility mechanisms and may be expected to occur although at unknown levels. Self-produced progenies are predicted to be less vigorous and thus, at high level selfing may affect the performance of a family at high level, hiding the breeding value of progenitors. The marker approach to quantify selfing levels in crosses is based on the identification or not of male-specific alleles in a sample of the progeny (McIntyre and Jackson, 2001, Euphytica 117: 245-249). A certain number of seedlings are genotyped with a set of loci and those seedlings without male-specific alleles are considered to be product of self-pollination.

Conventional techniques for the development of microsatellite markers are expensive and time-consuming, and generally require several steps (Cordeiro et al., 1999, Plant Mol. Biol. Rep. 17: 225-229).

A limited number of microsatellite markers are available for sugarcane in the specialized literature and in addition, the qualities of these markers are not fully validated for fingerprinting purposes (Aitken et al., 2005, Theor. Appl. Genet. 110: 789-801; Arro, 2005, In: M S Thesis Genetic Diversity Among Sugarcane Clones Using Target Region Amplification Polymorphism (Trap) Markers and Pedigree Relationships, Louisiana State University, Louisiana; Casu et al., 2005, Field Crops Res. 92: 137-147; Cordeiro, 2001, Molecular marker systems for sugarcane germplasm analysis In: Henry R J (ed.) Plant Genotyping—The DNA Fingerprinting of Plants,. CABI Publishing, Wallingford: 129-146; Cordeiro et al.; 1999, Plant Mol. Biol. Rep. 17: 225-229; Cordeiro et al., 2000, Plant Sci. 155: 161-168; Cordeiro et al., 2003, Plant Sci. 165: 181-189; Garcia et al., 2006, Theor. Appl. Genet. 112: 298-314; Jannoo et al, 2001, Proc. Intl Soc. Sugar Cane Technol. 24: 637-639; Pan et al., 2003, Maydica 48: 319-329; Pan et al.,2003, Plant and Animal Genome Abstract XI Abstract Book W189: 43; Pinto et al., 2004, Genome 47: 795-804).

The expressed genome of sugarcane represented by ESTs (Expressed Sequence Tags) has been sequenced and became a rich source of microsatellite markers (Silva, 2001, Gen. Mol. Biol. 24: 155-159; Vettore et al., 2001, Gen. Mol. Biol. 24: 1-7). The development of a molecular marker system stable enough to be reproducible at standard conditions and that discriminate individuals has been a major concern of sugarcane breeders (Cordeiro, 2001, Molecular marker systems for sugarcane germplasm analysis In: Henry R J (ed.) Plant Genotyping—The DNA Fingerprinting of Plants, CABI Publishing, Wallingford: 129-146).

### SUMMARY OF THE INVENTION

The present invention discloses methods and means for identification, evaluation and validation of microsatellites markers present in the genome of species from the Saccharum complex, particularly from the genus Saccharum obtained as Expressed Sequence Tags-sequences and the use of these markers for fingerprinting of Saccharum complex accessions.

It is an object of this invention providing methods and means for the selection of a set of Expressed Sequence Tags-derived microsatellite markers capable of discriminate a large number of the Saccharum complex accessions that share different levels of parentage, to (a) establish technical conditions for reproducibility of results after Polyacrylamide Gel Electrophoresis (PAGE) and DNA sequencer resolution, two popular and widespread techniques for the detection of DNA marker polymorphism; to (b) use the marker set as a tool in Saccharum complex breeding; and (c) as a candidate supporting system for property rights' issues.

The present invention also provides isolated microsatellite sequences obtainable from Saccharum spp., together with flanking sequences specific to the inventive microsatellite sequence. Methods for the use of probes and primers designed from such microsatellite and flanking sequences, together with kits comprising such probes and primers are also provided.

In a first aspect, the present invention provides isolated polynucleotides comprising at least one microsatellite repeat and at least two associated flanking sequences. In one embodiment, the isolated polynucleotides of the present invention comprise a sequence selected from the group consisting of: (i) sequences provided in SEQ ID NQ: 1-10; (ii) sequences complementary to sequences provided, in SEQ ID NO: 1-10; and (iii) variants of a sequence of (i) or (ii) having alterations in repeat number into a microsatellite.

In a second aspect the present invention provides primer pairs comprising a forward and a reverse primer for amplification of microsatellite located in DNA from a Saccharum species or related genera (Saccharum complex). Primer pairs suitable for polymerase chain reaction (PCR) amplification of microsatellite may be selected from the group consisting of SEQs ID NO: 11 -30.

In a third aspect the present invention provides a method for determining a genotype in a sample from the Saccharum complex. The method comprises the determination of the Saccharum complex genotype and a comparison of said scored results with results of analysis of DNA from predetermined known samples. With this method is possible to identify unknown samples. Methods for identification may be used for management of germplasm collections; for identification of hybrids between to distinct genotypes belonging or not to two different species; for identification of mislabelled plantlet samples produced by tissue culture procedures; for identification of varietals mixture of plant in sugarcane field; for identification and detection of the transgenes by multiplex of set markers with specific primers of inserted gene.

In a fourth aspect the present invention provides a method tbr introgression of desirable genomic sequences from one accession into another of samples from the Saccharum complex.

In a fifth aspect the present invention provides methods for selection of progenitors from the Saccharum complex by using the capability of microsatellites of the present invention to estimate the genetic similarity between progenitors.

In a sixth aspect the present invention provides methods for determination of the progenitor in cases where the father and/or the mother are unknown.

In a seventh aspect the present invention provides methods for quantification of self-pollinated in seedlings samples coming from a specific cross involving one or more Saccharum complex progenitors.

In an eighth aspect of the invention kits are herein provided for use with commercially available polymerase chain reaction (PCR) instruments to detect accessions of samples from the Saccharum complex.

In a ninth aspect the present invention provides methods to locate (mapping) genes linked to alleles of quantitative trait loci (QTLs) for the development of MAS (marker-assisted selection) procedures.

### BRIEF DESCRIPTION OF THE DRAWINGS

FIG. 1 shows the capacity of the simple sequence repeat (SSR) fingerprinting system to discriminate Saccharum complex accessions and related species sharing diverse levels of genetic relationship, as described in Example 5.

FIG. 2 shows the capacity of the simple sequence repeat (SSR) fingerprinting system to discriminate 48 selfing F1 progenies of variety RB855002, as described in Example 5.

FIG. 3 shows the paternity determination in polycrosses. RB966928 is the female parent and there are 30 possible male parents, 29 male parents plus the female parent which can act as male as well (self-pollination), as described in Example 7.

### DETAILED DESCRIPTION OF THE INVENTION

The present invention discloses methods and means for detecting and identifying accessions from the Saccharum complex by microsatellite analysis genotyping and identifying within the genome of the Saccharum complex. The present invention provides novel isolated microsatellite DNA sequences and DNA sequences flanking such microsatellites, such sequences being obtainable from Saccharum spp. accessions. Specifically, the present invention provides: (i) isolated polynucleotide sequence of SEQ ID NO: 1-10; and (ii) a complement of a sequence of SEQ ID No: 1-10; and (iii) a variant thereof.

In a first aspect, the present invention provides isolated polynucleotides comprising at least one microsatellite repeat and at least two associated flanking sequences. In one embodiment, the isolated polynucleotides of the present invention comprise a sequence selected from the group consisting of: (i) sequences provided in SEQ ID NO: 1-10;. (ii) sequences complementary to sequences provided in SEQ ID NO: 1-10; and (iii) variants of a sequence of (i) or (ii) having alterations in repeat number into a microsatellite

The isolated polynucleotide sequences of the present invention have utility in: (a) the identification of accessions from the Saccharum complex, (b) detection of DNA polymorphisms, (c) genome mapping, and (d) evaluation of genetic variability within and between plant tissues, populations, cultivars, species and species groups.

The isolated microsatellite repeats with their single copy flanking sequences provide locus-specific markers. The flanking sequences may be used to design locus-specific primers to amplify and detect the presence of the microsatellite sequence in a plant's genome.

The Saccharum complex plant DNA may be a genomic DNA or a cDNA. Generally, microsatellites are more frequently present in non-coding regions of the genome, but this does not preclude isolation of microsatellites which may be presented in transcribed regions, such as represented in mRNA and cDNA derived therefrom, which process is well understood in the art. In the present invention the DNA is, preferably, a cDNA.

A database of 352,122 Saccharum spp. Expressed Sequence Tags ESTs was screened for sequences with simple sequences repeats (SSR) motifs. The redundant database of 352,122 Saccharum spp. EST sequences (reads) was obtained by combining public available sequences from the SUCEST project (Vettore et al., 2001, Gen. Mol. Biol. 24: 1-7) and others sources that have been deposited in GenBank (Benson et al., 2006, Nucleic Acids Res. 34: 16-20) with a private initiative database (www.alellyx.com.br).

The redundant Expressed Sequence Tags EST database was screened for single and compound simple sequences repeats (SSR) motifs with a software for microsatellite identification, in this particular case the MISA software (Thiel et al., 2003, Theor. Appl. Genet. 106: 411-422). The program was set to identify repeat motifs with one (mono), two (di), three (tri), four (tetra), five (penta) and six (hexa) nucleotides in size. The below limit for number of repeat units in the motif was set to be at least ten repeats for mono, six for di and at least five for tetra, penta and hexa motifs. Compound motifs were defined as those with two or more repeat motifs separated by 100 bases or less.

The sub-database of identified simple sequences repeats (SSR)-containing Expressed Sequence Tags EST sequences was assembled to form clusters of redundant sequences as taught by Telles and Silva, 2001, Gen. Mol. Biol. 24: 17-23. Clusters were classified according to the type of simple sequences repeats (SSR) motif. Selection of candidate markers was performed separately for each type of motif and by two criteria: clusters with the highest number of reads differing in the number of repeat units (in silico polymorphism) and clusters with reads containing the highest number of repeat units (potential polymorphism).

In a second aspect of the invention, primer pairs comprising a forward and a reverse primer are presented for amplification of microsatellite located in DNA from accessions from the Saccharum complex. Primer pairs suitable for Polymerase Chain Reaction (PCR) amplification of microsatellite are selected from the group consisting of SEQ ID NO. 11-30.

A pair of primers was designed for each in silico-selected cluster with the aid of a program for designing polymerase chain reaction (PCR) primers, such as the software Primer3 (Rozen and Skaletsky, 1998, Primer3 on the WWW for general users for biologist programmers In: Krawetz S. and Misener S. (eds.) Bioinformatics Methods and Protocols: methods in molecular biology, Human Press, Totowa: 365-382). The program was set to identify, in the assembled consensus sequences, pairs of primers with melting temperature (Tm) around 64° C., 19-22 bases each in length, comprising sequences flanking the simple sequences repeats (SSR) motif (at least 5 bases away from the 5′ and 3′ limits of the motif) and to amplify fragments between 100 and 250 bp.

In a third aspect of the invention, a method for determining genotype in a sample from the Saccharum complexis presented. The method comprises the steps of: (i) obtaining DNA from the sample; (ii) amplifying simple sequence repeat (SSR) loci selected from the group consisting of: (i) sequences provided in SEQ ID NO: 1 -10; and (ii) sequences complementary to a sequence provided in SEQ ID NO: 1-10; with a set of primers in the DNA selected from the group consisting of: (i) sequences provided in SEQ ID NO: 11-30; (ii) sequences complementary to a sequence provided in SEQ ID NO: 11-30, and (iii) sequences comprising at least 6 contiguous nucleotides of a sequence provided in SEQ ID NO: 11-30 of primer pairs consisting of SEQ ID NO. 11-30 to form amplification products of various sizes and labels; (iii) separating amplification products by size; (iv) scoring the results of said separation; and (v) comparing said scored results to results of analysis of DNA from a known species that have been previously analysed and determined. This method allows the identification of accessions for linking a sample from the Saccharum complex to a plant source comprising the steps: (i) determining the identity of DNA in said sample by presented method, (ii) determining the identity of DNA in a sample from a known plant genotype by the presented method; and (iii) comparing the genotypes of both samples to determine the identities of sample (i).

Methods for identification determining genotype in a sample from Saccharum complex may be used for different applications, such as:
(a) Management of germplasm collections, grouping similar clones, eliminating duplicates and identifying mislabeling of accessions. The International Society of Sugarcane Technologists has identified fingerprinting of germplasm as an important issue;
(b) Identification of hybrids between to distinct genotypes belonging or not to two different species, genera;
(c) Certification for “quality control” through the identification of mislabeling of plantlet samples produced by tissue culture procedures;
(d) Identification of varietals mixture of plants from sugarcane field certifying nursery, and
(e) Identification and detection of the transgenes by multiplex of set markers with specific primers of inserted gene.

In a fourth aspect the present invention provides a method for introgression of desirable genomic sequences from one accession into other in samples from the Saccharum complex, identifying among offspring those hybrids that have alleles from both female and male parent utilized in the cross, using set markers. The method comprises the identification of female and male alleles utilizing the steps of: (i) obtaining DNA from the samples (such as: female and male parents and offspring), (ii) amplifying simple sequences repeats (SSR) loci with a set of primers in DNA selected from the group of primer pairs from SEQ ID NO. 11-30 to form amplification products of various sizes and labels; (iii) separating amplification products by size; (iv) identifying male and female parent exclusives alleles; (v) identifying offspring individuals with both male and female parent exclusives alleles (hybrid individuals). The results can be used to select hybrid individuals with desirable introgressed genomic sequences that may be used in forthcoming crosses.

In a fifth aspect the present invention provides a method for selection of progenitors by using the capability of microsatellites to estimate the genetic similarity between accessions. The method comprises the determination of genotypes for a selected group of samples from the Saccharum complex, which will be used as parents in crosses and a comparison of a said scored results with the aid of specific genetic similarity estimation softwares (NTSys for instance). The results of this analysis will provide indices of genetic similarity between accessions which will be used to drive biparent crosses (one male and one female parent) with higher probability of generating improved offspring (heterosis). The method comprises the determination of genotypes for a selected group of samples from the Saccharum complex utilizing the steps of: (i) obtaining DNA from the samples (such as a group of sugarcane accessions), (ii) amplifying simple sequences repeats (SSR) loci with a set of primers in DNA selected from the group of primer pairs form SEQ ID NO. 11-30 to form amplification products of various sizes and labels; (iii) separating amplification products by size; (iv) generating a microsatellite binary data with the scored results; and (v) performing a mathematical treatment of the binary data containing the scored results with the aid of specific genetic similarity estimation softwares for generating parent selection indices. The resulting indices can be used to select parents to be used in crosses.

Estimation of genetic diversity in Saccharum complex breeding populations is used to produce genetic distance indices with which breeders may predict heterosis of parents and performance of progenies (Lima et al., 2002, Theor. Appl. Genet. 104: 30-38). These indices are readily calculated with the microsatellite binary data produced after fingerprinting a breeding population and have been used to orientate parent selection. Prediction of heterosis allows reduction in the number of crosses and progenies to be screened and have a direct effect in the costs and time of development of a new variety. The same effects are expected to come from the utilization of this microsatellite set and other DNA markers in the mapping of important genes and traits and the subsequent development of marker assisted selection schemes (McIntyre et al., 2005, Mol. Breed. 16: 151-161).

In a sixth aspect the present invention provides a method for determining paternity of accessions from the Saccharum complex, in cases where the father and/or the mother are unknown. The method comprises the determination of genotypes of the Saccharum complex (offspring) and a comparison of said scored results with results of analysis of DNA from the female parent and possible male parent accessions. The results will compare firstly the offspring alleles with the female parent exclusives alleles. Once the female parent alleles are identified, the rest of alleles in the offspring are male specific. A search is performed to identify in the possible male parents which one has all male specific alleles and that should be the true male parent of the offspring. The identification of a parent of a Saccharum complex genotype comprises the steps of: (i) obtaining DNA from the accession (such as: offspring, known female parent and possible male parents), (ii) amplifying simple sequences repeats (SSR) loci with a set of primers in said DNA selected from the group of primer pairs from SEQ ID NO. 11-30 to form amplification products of various sizes and labels; (iii) separating amplification products by size; (iv) identification of female and male specific alleles; and (v) identification of true male parent.

In a seventh aspect the present invention provides a method for quantification of self-pollination in seedlings samples from the Saccharum complex coming from a specific cross involving one or more progenitors. The method comprises the determination of genotypes of the Saccharum complex and a comparison of the scored results with results of analysis of DNA from parents accessions. The results will compare the alleles of the offspring with those exclusives from the female and male parents. Offspring identified as not containing male exclusives alleles are products of self-pollination. By performing this analysis in several offspring, it is possible to calculate the percentage of self-pollination in a seedling sample from a specific cross. The determination of self-pollination in a seedling sample comprises the steps of: (i) obtaining DNA from the samples (such as offspring and parents), (ii) amplifying simple sequences repeats (SSR) loci with a set of primers in DNA selected from the group of primer pairs from SEQ ID NO. 11-30 to form amplification products of various sizes and labels; (iii) separating amplification products by size; (iv) identifying female and male specific alleles; and (v) identifying the offspring that contain only female parent exclusives alleles.

In an eighth aspect of the invention, kits are provided for use with Polymerase Chain Reaction (PCR) instruments to detect accessions from the Saccharum complex. The kits comprise one or more primer pairs suitable for amplifying a nucleotide sequence in a sample of the complex by Polymerase Chain Reaction, wherein the nucleotide sequence comprises a sequence from the group consisting of SEQ ID No 1-10. Preferably the kits comprise primer pairs having SEQ ID NO: 11-30. The kits may utilize a multiplex methodology.

The kits may further comprise primers, enzymes, Taq polymerases, for example, salts and buffers suitable for causing amplification by Polymerase Chain Reaction. The kits also comprise preferably a positive control. In certain preferred embodiments of the kit the primers comprise a label whereby amplified microsatellites may be detected. In other preferred embodiments of the kit, labeled nucleic acids are provided. Observable labels are preferably fluorescent molecules or radionucleotides. The kits may also comprise suitable containers and bottles for housing these reagents and or convenient use.

In a ninth aspect the present invention provides a method for localization (mapping) of genes and quantitative trait loci (QTLs) for the development of MAS (marker-assisted selection) procedures. Marker-assisted selection (MAS) is based on the concept that it is possible to infer the presence of a gene from the presence of a marker that is tightly linked to the gene. If the marker and the gene are located far apart then the possibility that they will be transmitted together to the progeny individuals will be reduced due to double crossover recombination events. Hence a prerequisite to using markers in such selection is that they should be tightly linked to the gene of interest. For this purpose, saturation of regions (encompassing the locus of interest) on the genetic linkage map is necessary. Saturation is a relative term and the degree of saturation depends upon the purpose for which the map is required. The degree of saturation is the proportion of the genome that will be covered by markers at a density such that the maximum separation between markers is no greater than some small number of centimorgans.

Definitions

As used herein “microsatellites”, also referred to as simple sequences, simple sequences repeats (SSRs), simple repetitive DNA sequences, short tandem repeats (STRs) or simple sequences motifs (SSMs) are stretches of DNA found in coding and non-coding areas of genomes of every living organisms. They are composed of number of tandemly repeated short nucleotide motifs or repeats units. Microsatellites are formed by one to six nucleotide units repeated in tandem, varying in length between genomic chromosome copies. Microsatellites are highly polymorphic.

As used herein, the term “flanking sequence” refers to the non-repetitive, nucleotide sequence adjacent to a microsatellite. “Unique flanking sequences” are those flanking sequences which are only found at one location within the genome.

As used herein, “accessions” refers to individuals of any species, genus, hybrids interspecific, hybrids intergenus into Saccharum complex.

Saccharum complex is all species of genus Saccharum with species of genus Erianthus and Miscanthus, Sclerostachya and Narenga, genus phylogenetically related to Saccharum spp..

As used herein, the term “polynucleotide” includes DNA and RNA molecules, both sense and anti-sense strands, and comprehends cDNA, genomic DNA, recombinant DNA and wholly or partially synthesized polynucleotide. All the polynucleotides provided by the present invention are isolated and purified, as those terms are commonly used in the art.

As used herein, the term “primer” is a single-stranded oligonucleotide which is capable of acting as an initiation point for synthesis of either DNA or RNA from a specific sequence when placed under conditions which induce synthesis of a primer extension product complementary. “Primer pair” is two primers including primer 1 that hybridizes to a single strand at one end of the DNA sequence to be amplified and primer 2 that hybridizes with the other end on the complementary strand of the DNA sequence to be amplified.

A “primer pair” is selected to detect a specific microsatellite. Each primer of each pair is selected to be complementary to a different strand in the flanking sequence or a variant of a flanking sequence of each specific microsatellite sequence to be amplified. Although the primer sequence need not reflect the exact sequence of the naturally occurring flanking sequence, the more closely the 3′ end reflects the exact sequence, the better the binding during the annealing stage. Differential labels may be employed, as describe for example in U.S. Pat. No. 5,364,759, to Caskey et al. to distinguish extension products from each other.

“Polymerase chain reaction” or “PCR” is a technique in which cycles of denaturation, annealing with primer, and extension with DNA polymerase are used to amplify the number of copies of a target DNA sequence by approximately 106 times or more. The polymerase chain reaction process for amplifying nucleic acid is covered by U.S. Pat. No. 4,683,195 to Mullis et al. and U.S. Pat. No. 4,683,202 to Mullis, which are incorporated herein by reference for a description of the process.

As used herein, the term “variant of a sequence” comprehends sequences having alterations in repeat number into a microsatellite. By primers variants is meant having base deletions, additions or substitutions which do not alter the ability of the primer to be useful in amplifying microsatellites.

As used herein, “allele” is a genetic variation associated with a segment of DNA, i.e., one of two or more alternate forms of a DNA sequence occupying the same locus.

“DNA polymorphism” is the condition in which two or more different nucleotide sequences in a DNA sequence coexist in the same interbreeding population.

“Polymorphism information content” or “PIC” is a measure of the amount of polymorphism present at a locus (Botstein et al., 1980, Am. J. Hum. Genet. 32: 314-331). PIC values range from 0 to 1.0, with higher values indicating greater degrees of polymorphism. This measure generally displays smaller values than the other commonly used measure, i.e., heterozygosities. For markers that are highly informative (heterozygosities exceeding about 70%), the difference between heterozygosity and PIC is slight.

All technical terms used herein are terms commonly used in biochemistry, molecular biology and agriculture, and can be understood by one of ordinary skill in the art to which this invention belongs. Those technical terms can be found in: Molecular Cloning: A Laboratory Manual, 3rd ed., vol. 1-3, ed. Sambrook and Russel, Cold Spring Harbor Laboratory Press, Cold Spring Harbor, N.Y., 2001; Current Protocols in Molecular Biology, ed. Ausubel et al., Greene Publishing Associates and Wiley-Interscience, New York, 1988 (with periodic updates); Short Protocols in Molecular Biology: A Compendium of Methods from Current Protocols in Molecular Biology, 5th ed., vol. 1-2, ed. Ausubel et al., John Wiley & Sons, Inc., 2002; Genome Analysis: A Laboratory Manual, vol. 1-2, ed. Green et al., Cold Spring Harbor Laboratory Press, Cold Spring Harbor, N.Y., 1997. Methods involving plant biology techniques are described herein and are described in detail in methodology treatises such as Methods in Plant Molecular Biology: A Laboratory Course Manual, ed. Maliga et al., Cold Spring Harbor Laboratory Press, Cold Spring Harbor, N.Y., 1995. Various techniques using PCR are described, e.g., in Innis et al., PCR Protocols: A Guide to Methods and Applications, Academic Press, San Diego, 1990 and in Dieffenbach and Dveksler, PCR Primer: A Laboratory Manual, 2nd ed., Cold Spring Harbor Laboratory Press, Cold Spring Harbor, N.Y., 2003.

The present invention is further illustrated by the following specific examples. The examples are provided for illustration only and are not to be construed as limiting the scope or content of the invention in any way.

### EXAMPLES

### Example 1

Identification of Saccharum spp. microsatellite repeat sequences

Database of Saccharum spp. EST sequences

The redundant database of 352,122 Saccharum spp. EST sequences (reads) was obtained by combining public available sequences from the SUCEST. project (Vettore et al., 2001, Gen. Mol. Biol. 24: 1-7) and others sources that have been deposited in GenBank (Benson et al., 2006, Nucleic Acids Res. 34: 16-20) with a private initiative database (www.alellyx.com.br).

In silico selection of simple sequence repeat (SSR) markers

The redundant EST database was screened for single and compound simple sequence repeat (SSR) motifs with a software for microsatellite identification, in this particular case MISA software (Thiel et al., 2003, Theor. Appl. Genet. 106: 411-422) and the results of this search are summarized in Table 1.

The software identified 36,950 simple sequence repeat (SSR) motifs and retrieved 33,324 redundant sequences (9.46%). The database was assembled into 79,526 clusters of which 14,291 contain simple sequence repeat SSR motifs (18%). Of these clusters, 10,586 (75%) had a single motif and 3,705 had two or more. Table 2 shows the distribution of the 36,950 SSR-containing redundant sequences classified according to the type of motif. The most abundant unit size motifs were tri (48%), followed by mono (32%), and di (16%). Tetra, penta, and hexa motifs were identified in 1,414 sequences, representing less than 5% of the SSR-containing database sequences.

To select sequences for the development of simple sequence repeat (SSR) markers, the 36,950 SSR-containing redundant sequences were organized in a sub-database along with information concerning read and cluster names, and repeat unit size, number, and sequence. This sub-database of candidate simple sequence repeat SSR markers was sorted initially by repeat unit size and divided into seven subsets of reads (compound, mono, di, tri, tetra, penta, and hexa motifs). Sequences with mono and di motifs were not used further in marker selection due to our own experience with the low resolution of these unit repeats in Polyacrylamide Gel Electrophoresis (PAGE) and DNA sequencer. The five remain subsets of reads were treated separately as follow. Each subset was grouped by cluster name. Inspection of reads belonging to the same cluster revealed sequences sharing motifs with the same unit size and sequence but varying in the number of repeat units. This polymorphism observed in silico was elected as a selection criterion. Each subset was then sorted for clusters with the highest number of reads differing in the number of repeat units. The top ranked clusters were checked individually for enough 5′ and 3′ sequences to design primers and 63 consensus sequences were selected for further characterization. A second round of in silico selection involved the sorting of subsets by clusters containing reads with the largest number of repeat units and thus, with potentiality for having a large number of alleles. At this time clusters poorly represented by reads and that may be missed in the first round of selection were well ranked. The top clusters were checked for enough 5′ and 3′ sequences and 49 consensus sequences were selected for further characterization. A total of 112 clusters were selected for the design of primers and further evaluation by a validation process. Table 3 summarized the results of the in silico selection of SSR-containing clusters performed under two selection criteria.

Primer Design.

A pair of primers was designed for each in silico-selected cluster with the aid of a program for designing polymerase chain reaction, in this case the software Primer3 (Rozen and Skaletsky, 1998, Primer3 on the WWW for general users for biologist programmers In: Krawetz S. and Misener S. (eds.) Bioinformatics Methods and Protocols: methods in molecular biology, Human Press, Totowa: 365-382). The program was set to identify, in the assembled consensus sequences, pairs of primers with melting temperature (Tm) around 64° C., 19-22 bases each in length, comprising sequences flanking the SRR motif (at least 5 bases away from the 5′ and 3′ limits of the motif) and to amplify fragments between 100 and 250 bp. The sequences are described in the Sequence Listing, according to Table 4.

TABLE 1
Summarized results of the search for SSR motifs in the
Saccharum spp. EST database.

| Singlet sequences | Total number | Frequency (%) |
|---|---|---|
| Databank singlet sequences | 352,122 | 100% |
| Singlet sequences with SSR | 33,324 | 9.46% |
| Identified SSR motifs | 36,950 | 10.49% |
| Singlets with >1 motif | 2,936 | 0.83% |
| Compound SSR motifs | 2,394 | 0.68% |

| Assembled sequences | Total number | Frequency (%)* |
|---|---|---|
| Assembled sequences (clusters) | 79,526 | 100% |
| Assembled sequences with SSR | 14,291 | 17.97% (100%) |
| Assembled sequences with 1 SSR | 10,586 | 13.31% (75%) |
| Assembled sequences with 2 SSR | 2,185 | 2.74% (15%) |
| Assembled sequences with 3 SSR | 737 | 0.93% (5%) |
| Assembled sequences with >3 SSR | 783 | 0.98% (5%) |

*Values between parentheses referred to the distribution of SSR-containing assembled sequences according to the number of motifs per sequence.

TABLE 2
Distribution of SSR motifs by unit size in the Saccharum spp.
EST singlet sequence database.

| Type of motifs by unit size | Sequences | Frequency (%) |
|---|---|---|
| All types | 36,950 | 100% |
| 1 (mono) | 11,978 | 32.42% |
| 2 (di) | 5,846 | 15.82% |
| 3 (tri) | 17,712 | 47.93% |
| 4 (tetra) | 644 | 1.74% |
| 5 (penta) | 527 | 1.42% |
| 6 (hexa) | 243 | 0.66% |

TABLE 3
Selection of 112 clusters by “in silico polymorphism” and “potential
polymorphism” criteria.

| | | Selection criteria | |
|---|---|---|---|
| Motif type | Selected clusters | Number of alleles | Number of repeats |
| Compound | 12 | 5 (42%) | 7 (58%) |
| Tri | 45 | 33 (73%) | 12 (27%) |
| Tetra | 29 | 14 (48%) | 15 (52%) |
| Penta | 13 | 4 (31%) | 9 (69%) |
| Hexa | 13 | 7 (54%) | 6 (46%) |
| Total | 112 | 63 (56%) | 49 (44%) |

TABLE 4
Relationship between microsatellites and primers with sequences
describe in Sequence listing.

| Locus | Sequence | Primer F | Primer R |
|---|---|---|---|
| CV22 | SEQ ID NO 1 | SEQ ID NO 11 | SEQ ID NO 12 |
| CV29 | SEQ ID NO 2 | SEQ ID NO 13 | SEQ ID NO 14 |
| CV37 | SEQ ID NO 3 | SEQ ID NO 15 | SEQ ID NO 16 |
| CV38 | SEQ ID NO 4 | SEQ ID NO 17 | SEQ ID NO 18 |
| CV60 | SEQ ID NO 5 | SEQ ID NO 19 | SEQ ID NO 20 |
| CV78 | SEQ ID NO 6 | SEQ ID NO 21 | SEQ ID NO 22 |
| CV79 | SEQ ID NO 7 | SEQ ID NO 23 | SEQ ID NO 24 |
| CV94 | SEQ ID NO 8 | SEQ ID NO 25 | SEQ ID NO 26 |
| CV104 | SEQ ID NO 9 | SEQ ID NO 27 | SEQ ID NO 28 |
| CV106 | SEQ ID NO 10 | SEQ ID NO 29 | SEQ ID NO 30 |

### Example 2

Selection of a set of SSR-containing loci for Saccharum complex fingerprinting in Polyacrylamide Gel Electrophoresis (PAGE) and DNA sequencer systems after validation in silico.

A two-stage validation process was setup with the goal of selecting ten pairs of primers (loci) suitable for Saccharum complex fingerprinting in both Polyacrylamide Gel Electrophoresis (PAGE) and DNA fragment detection systems. The first stage (eliminatory) involved the amplification of DNA from four Saccharum complex accessions (Caiana, Q136, RB835054, and RB855036, Table 5) with all selected pairs of primers and resolution of fragments in Polyacrylamide Gel Electrophoresis (PAGE). Profiles were inspected and scored according to criteria regarding visual quality of amplified profiles (fragments within the expected size range and intense and resolved enough to be scored), polymorphism of fragments (at least 4 fragments of different sizes) and low incidence of PCR (polymerase chain reaction) artifacts such as stutters. Polymerase chain reactions and Polyacrylamide Gel Electrophoresis (PAGE) were done three times before excluding a candidate marker (pair of primers). Data shown in Table 6.

Overall, inadequate quality of profiles (polymerase chain reactions quality and artifact criteria) was responsible for the elimination of 57% of the candidate loci (64) whereas low polymorphism responded for 25% (28). A total of 20 loci (18%) with representatives of the five types of simple sequence repeat (SSR) motifs were selected at the end of this validation stage.

The second stage of the validation process (classificatory) involved the amplification of DNA from 27 Saccharum complex accessions (Table 5) with the 20 previous selected pairs of primers (loci) in the first stage followed by resolution. The amplification reactions were resolved in Polyacrylamide Gel Electrophoresis (PAGE) and DNA sequencer (with forward primers labeled with fluorescent dyes), evaluated and ranked according to three criteria: quality of PCR (polymerase chain reaction) reaction (fragments within the expected size range and intense and resolved enough to be scored); polymorphism of the profiles (at least 6 fragments of different sizes with high PIC value) and low incidence of PCR artifacts such as stutters. The PIC value for each locus was calculated (Table 7) from the total number and frequencies of alleles in 27 Saccharum complex accessions used for validation. Polymorphism information content (PIC) values were sufficient high for all loci (0.774-0.918) and thus, visual quality of profiles was a mandatory criterion for classification. After profiles have been evaluated and ranked, the top ten loci were selected to compose a simple sequence repeat-based fingerprinting system.

Data Scoring and Analysis.

Alleles for each accession were visualized as bands (Polyacrylamide Gel Electrophoresis—PAGE) or peaks (DNA sequencer) and scored as present (1) or absent (0) manually by at least two analysts. Values for polymorphism information content (PIC) of markers were calculated from the formula below (Weber, 1990, Genomics 7: 524-530; Anderson et al., 1993, Genome 36: 181-186) where Pij is the frequency of the jth allele in genotype (i). Scored alleles were entered into a binary data matrix as discrete variables.

Cluster analyses were performed using the software for determinate genetic similarities, particularly the NTSYSpc version 2.20 (Rohlf, 2002, NTSYSpc: Numerical Taxonomy System, ver. 2.2, Exeter Publishing Ltd., Setauket, N.Y.). Similarities between accessions were estimated with the Jaccard's coefficient (Sneath and Sokal, 1973, Numerical Taxonomy: the principles and practice of numerical classification, Freeman, San Francisco, 573p) and clusters were depicted on estimated similarities calculated with the unweighted pair group method with arithmetic mean (UPGMA). The Simple Matching (SM) coefficient (Sokal and Michener, 1958, Univ. Kansas Sci. Bull. 38: 1409-1438) was used to calculate the number of alleles that discriminate two accessions [(1-SM)×total number of alleles=number of discriminatory alleles]. The Probability of Genetic Identity (Pid) which represents the probability that two randomly selected individuals will share the same genotype was calculated using the formula Pid=Σχ2, where χ is the genotypic frequency (Paetkau et al., 1995, Mol. Ecol. 4: 347-358). Pid for combinations of loci was calculated by multiplying the corresponding Pid values of each locus involved.

TABLE 5
List of 27 Saccharum spp. accessions fingerprinted in the second
stage of the SSR marker validation process and the corresponding
immediate pedigrees.

| Accession* | Female parent | Male parent |
|---|---|---|
| Caiana (S. officinarum) | Unknown | Unknown |
| Co740 | P3247 | P4775 |
| IAC86-2210 | CP52-48 | Co798 |
| Olhuda (S. officinarum) | Unknown | Unknown |
| Preta Kavangire (S. officinarum) | Unknown | Unknown |
| Q136 | NCo310 | 54N7096 |
| RB72454 | CP53-76 | Unknown |
| RB835054 | RB72454 | NA56-79 |
| RB835486 | L60-14 | Unknown |
| RB855036 | RB72454 | SP70-1143 |
| RB855453 | TUC71-7 | Unknown |
| RB855536 | RB72454 | SP70-1143 |
| RB867515 | RB72454 | Unknown |
| RB925345 | H59-1966 | Unknown |
| RB928064 | SP70-1143 | Unknown |
| S1 | RB835054 | RB855036 |
| S2 | RB835054 | RB855036 |
| S3 | RB835054 | RB855036 |
| S4 | RB835054 | RB855036 |
| S5 | RB835054 | RB855036 |
| S6 | RB835054 | RB855036 |
| S7 | RB835054 | RB855036 |
| S8 | RB835054 | RB855036 |
| SP80-1842 | SP71-1088 | H57-5028 |
| SP81-3250 | CP70-1547 | SP71-1279 |
| SP83-2847 | HJ5741 | SP70-1143 |
| SP89-1115 | CP73-1547 | Unknown |

*Accessions S1 to S8 are F1 progenies from a cross between half-sib varieties RB835054 and RB855036.

TABLE 6
Summarized results of the first stage of the SSR marker validation
process showing the selection of 20 loci among 112 candidates.

| | Elimination criteria* | | | |
|---|---|---|---|---|
| Motif type | PCR | Number of | | Selected loci |
| (number of loci) | quality | alleles | PCR artifact | Total number |
| Compound (12) | 5 (41%) | 2 (17%) | 3 (25%) | 2 (17%) |
| Tri (45) | 23 (51%) | 11 (24%) | 5 (11%) | 6 (14%) |
| Tetra (29) | 13 (45%) | 9 (31%) | 2 (7%) | 5 (17%) |
| Penta (13) | 3 (23%) | 4 (31%) | 3 (23%) | 3 (23%) |
| Hexa (13) | 7 (53%) | 2 (16%) | 0 (0%) | 4 (31%) |
| Total (112) | 51 (46%) | 28 (25%) | 13 (11%) | 20 (18%) |

*Elimination by PCR quality includes no amplification, low amplification and amplification of fragments out of the expected size range. PCR artifacts refer mainly to the frequency and intensity of stutters. Number of alleles refers to monomorphic profiles or combined profiles with less than four different fragments.

TABLE 7
Summarized results of the second stage of the SSR marker validation
process showing the top 10 loci selected to form a Saccharum
complex fingerprinting system.

| | | Classificatory criteria* | | | |
|---|---|---|---|---|---|
| Loci | Motif type | Resolution | Artifact | PIC value | Status |
| CV29 | Tetra | +++ | +++ | 0.91 | Selected |
| CV38 | Penta | +++ | +++ | 0.90 | Selected |
| CV37 | Tetra | +++ | +++ | 0.87 | Selected |
| CV106 | Tri | +++ | +++ | 0.83 | Selected |
| CV104 | Penta | +++ | +++ | 0.82 | Selected |
| CV60 | Hexa | +++ | +++ | 0.76 | Selected |
| CV79 | Hexa | +++ | +++ | 0.75 | Selected |
| CV94 | Compound | +++ | +++ | 0.74 | Selected |
| CV78 | Penta | +++ | ++ | 0.89 | Selected |
| CV22 | Tetra | +++ | ++ | 0.80 | Selected |
| CV133 | Tri | +++ | ++ | 0.82 | Not selected |
| CV98 | Tri | +++ | ++ | 0.80 | Not selected |
| CV128 | Tri | +++ | ++ | 0.79 | Not selected |
| CV80 | Tetra | ++ | +++ | 0.81 | Not selected |
| CV86 | Tetra | ++ | +++ | 0.80 | Not selected |
| CV58 | Hexa | ++ | ++ | 0.89 | Not selected |
| CV140 | Hexa | ++ | ++ | 0.84 | Not selected |
| CV135 | Tri | ++ | ++ | 0.83 | Not selected |
| CV51 | Tri | ++ | ++ | 0.75 | Not selected |
| CV23 | Compound | ++ | + | 0.91 | Not selected |

*The following notes were used to evaluate fragment resolution and intensity of artifacts in both Polyacrylamide Gel Electrophoresis (PAGE) and DNA sequencer systems: + (satisfactory/high); ++ (good/medium) and +++ (excellent/low).
Classificatory criteria in order of importance are: resolution>artifact> polymorphism information content PIC value.
Polymorphism information content PIC values were calculated with number of alleles and frequencies observed in the 27 accessions used for validation.

### Example 3

Genotyping using microsatellites Saccharum spp. markers selected Plant material and tissue samples

A collection of 1,205 Saccharum complex accessions and related species originated from several national and international breeding programs and germplasm banks were selected for use in the present work (Table 8). Progenies of selfing (self-pollinated) and interspecific crosses and in, vitro plantlets of Brazilian commercial varieties were produced by the company's breeding station and tissue culture facility, respectively. Transgenic plants regenerated from calli were provided by Alellyx Applied Genomics (Campinas, São Paulo, Brazil). Field grown plants propagated by conventional seedcane were provided by mills located in the major production areas of Brazil (Northeast and Central-Southem States). To ensure the most homogeneous material, tissue samples were collected and send for analysis orientated by a standardized protocol. To avoid mislabeling and to have traceability, samples were stored in plastic bags or tubes identified by code bar labels.

This genotyping method may be applied for identification of all accessions and related species originated in the world

DNA Extraction Procedures

Total DNA was extracted from leaves, roots and buds of Saccharum complex and related species. Large scale preparations were carried out as follow. Approximately 100 mg of tissue powder (grinded with liquid N2) was suspended in 700 μL of extraction buffer (100 mM Tris-HCl, pH 8.0, 20 mM EDTA, 2% CTAB, 1% PVP 40, 1.4 M NaCl, 0.3% 2-mercaptoethanol, 50 μg RNase A). The suspension was fuilly mixed and incubated at 65° C. for 30 min. Total DNA was extracted with 800 μL of chloroform/isoamyl alcohol (24:1), the supernatant (approx. 600 μL) was recovered after centrifugation (12,000 g for 10 min. at room temperature), amended with 60 μL of extraction solution (10% CTAB, 1.4 M NaCl) and fully mixed. Total DNA was extracted as described above and precipitated with 450 μL of isopropanol. After DNA recovery by centrifugation (12,000 g for 5 min. at room temperature), the pellet was washed with 1 mL of ethanol 70%, dried at room temperature and solubilized in 100 μL of TE buffer (10 mM Tris-HCl, pH 8.0, 1 mM EDTA). DNA concentration was measured in spectrophotometer and adjusted to a standard 10 ng/μL solution.

For a high throughput processing of large numbers of samples, a 96 sample-platform was developed and mini prep extraction was carried out as described above with modifications. Plant material (usually 100-200 mg of young leaves) was introduced into wells of a 96 deep-well microplate (SSI). Each well received 200 μL of extraction buffer. A metal device with 96 pins was introduced into the wells and used to crush 30 times the plant material. An additional 300 μL of extraction buffer was dispensed into the wells and the plant material was crushed ten additional times. The plate was sealed with a rubber lid and left at 65° C. for 30 min, followed by the addition of 500 μL of chloroform/isoamyl alcohol (24:-1). The plate was vortex for 30 s and centrifuige at 3,200 g for 30 min at 4° C. The supernatant (˜200 μL) was transferred to another deep-well microplate, 20 μL of extraction solution was added and the samples were extracted with chloroform/isoamyl alcohol as described above. The supernatant (80 μL) was transferred to a 96 conventional microplate and 80 μL of isopropanol was added. The microplate was sealed, left at room temperature for 20 min and total DNA was recovered by centrifugation (3,200 g for 15 min at 4° C.). Pellets were washed with 150 μL of 70% ethanol, dried at room temperature and solubilized in 25 μL of TE buffer. Measurements in spectrophotometer showed a typical DNA concentration of 30 ng/μL.

Polymerase Chain Reaction PCR Amplification

Amplification of simple sequence repeat (SSR) markers was performed in 10 μL of reaction containing 2 μL of DNA (20 ng for large scale preps and 60 ng for mini scale preps), 4 μL of a reaction mix (50 units/mL Taq DNA Polimerase, 400 μM dATP, 400 μM dCTP, 400 μM dGTP, 400 μM dTTP, 100 mM Tris-HCl, pH9.0, 100 mM NaCl, 5 mM MgCl12, 0.2% IGepal), 0.5 μL of each primer (10 μM) and 3 μL of water. Reactions to be resolved in DNA sequencer were amplified with forward primers labeled with fluorescent dyes (ABI). Amplification was performed in thermocycler programmed as follow. An initial denaturation of 5 min at 94° C., 35 cycles of 94° C. for 30 s, 64° C. for 30 s and 72° C. for 30 s and a final extension at 72° C. for 60 min.

Fragment Detection

Fragments were resolved in Polyacrylamide Gel Electrophoresis (PAGE) and automated DNA sequencer systems. Denaturating polyacrylamide gels were made with 6% polyacrylamide (19:1), and 7 M urea in 0.5×TBE buffer (Sambrook et al., 1989, Molecular Cloning: a Laboratory Manual, 2^{nd} edition, Cold Spring Harbour Laboratory Press, New York). Samples were prepared with 6 μL of PCR reaction, 3 μL formamide and 1 μL loading buffer (formamide, 1 mM EDTA, 0.01% bromophenol blue and 0.01% xylene cyanol), denaturated at 95° C. for 5 min, kept on ice and loaded into the wells of the pre-heated gel (60 min. at 120 W). Samples were run for 3 h at 90 W and fragments were visualized by silver staining. Gels were immerse in a fixation solution (10% ethanol, 5% acetic acid) for 10 min., rinsed with deionized water, immerse in a pre-treatment solution (1.5% nitric acid) for 3 min., rinsed with deionized water, immerse in a 0.3% silver nitrate solution for 30 min., rinsed in deionized water twice, immerse in a developing solution (283 mM Na2CO3) for 15-30 min. or until fragments can be visualized. Scoring was done by visual inspection and size of fragments was estimated by comparison with size standard 25 bp Ladder (Promega). Samples amplified with labeled primers were resolved in an ABI3730 XL DNA Sequencer, with a 50 cm capillary array, loaded with POP7 polymer, set for 10 s injection time at 8.5 kV, electrophoresed at 8.5 kV and oven temperature of 60° C., and running time of 2 h. Fragment data generated by the sequencer were visualized with the software STRand (Hughes, 1998, Regents of the University of California, Davis, http://www.vgl.ucdavis.edu/STRand). Scoring was done by visual inspection and the size of fragments was estimated by comparison with size standard GeneScan 500 Liz (ABI) and with a proprietary size standard labeled with 6-FAM or NED.

TABLE 8
Collection of 1,205 accessions of Saccharum complex varieties, CV
clones under selection (CanaVialis program), and related species
classified by sign call (breeding program) and country of origin.

| Sign/species | Number of accessions | Country |
|---|---|---|
| B | 1 | Barbados |
| CB | 3 | Brazil |
| CC | 2 | Colombia |
| Co | 5 | India |
| CP | 10 | USA |
| CV | 320 | Brazil |
| IAC/IACSP | 19 | Brazil |
| LCP | 1 | USA |
| MZC | 1 | Cuba |
| NA | 1 | Argentina |
| PAV | 1 | Brazil |
| PO | 4 | Brazil |
| PR | 1 | Puerto Rico |
| Q | 4 | Australia |
| RB | 514 | Brazil |
| SP | 298 | Brazil |
| TUC | 1 | Argentina |
| VAT | 3 | Brazil |
| Saccharum officinarum | 11 | — |
| S. spontaneum | 1 | — |
| S. barberi | 1 | — |
| S. sinense | 1 | — |
| S. edule | 1 | — |
| Erianthus bengalensis | 1 | — |
| Total | 1,205 | 9 countries |

### Example 4

Characterization of alleles in the fingerprinting system after genotyping a collection of 1,205 Saccharum complex accessions and related species

A collection of 1,205 accessions of Saccharum complex varieties, clones under selection (CV program), and related species having worldwide origins and sharing diverse levels of genetic relationship (Table 8) were fingerprinted at the ten selected loci using the DNA sequencer detection system (Table 9). A total of 150 alleles were identified (average of 15 per locus) and their sizes were estimated with the aid of two distinct size standards. Locus CV38 revealed the highest number of alleles (27) followed by CV78 (25). CV29, CV37, CV22, CV79, and CV94 showed alleles in the range of 14-17 whereas CV 104, CV106, and CV60 in the range of 7-8 alleles. Frequencies of alleles in the fingerprinted population demonstrated great variability within and among loci. Observed polymorphism information content PIC values (0.77-0.92) were positively correlated with number of alleles. As judging by the differences in allele sizes within any particular locus, none of the selected loci displayed a perfect ladder of fragments expected from their respective repeat units. Locus CV22 for an example was selected as a perfect tetranucleotide repeat motif from its cluster consensus sequence but showed fragments differing in two, three, and five nucleotides. Carefully inspection of each consensus sequence used for primer design revealed several short runs of mono and di repeats located between the primer sites and the software MISA-identified simple sequence repeat (SSR) motifs. Such short motifs were under the threshold of the software but may also be polymorphic and thus, they would be responsible for the imperfections observed in the allele ladders. Sequencing of fragments corresponding to the alleles will be required to answer this question. Nevertheless, although troublesome for automated fragment scoring, theses imperfections create more variability (polymorphism) and thus, increase the discriminatory capacity of each locus.

Some inconsistencies related to certain alleles were observed after fingerprinting the same accessions more than once. Mapping of these unstable positions in the chromatograms revealed eight alleles distributed in loci CV37 (2), CV38 (4), and CV78 (2). Most of these alleles had consistently low PCR amplification in respect to the others alleles in the same locus and were frequently associated with positions where stutters commonly appear. This type of allele could be present in a certain genotype but would be missed during scoring in response to small fluctuations in the yields of PCR reactions. On the other hand, stutters that appear in a low amplification allele position were frequently and erroneously scored as true alleles. Due to the error prone nature of these alleles, further analyses of the data generated by the set of loci in the fingerprinted collection was carried out with 142 alleles (eight low amplification alleles were not considered).

TABLE 9
Characterization of alleles belonging to the SSR fingerprinting system in a collection of
1,205 accessions of Saccharum complex varieties and related species and mapping
of unstable positions^{a}.

| | Motif | | | | | | | | | |
|---|---|---|---|---|---|---|---|---|---|---|
| | | | | | | | | | | Comp. |
| | Tri | Tetra | | | Penta | | | Hexa | | (AAAAAG)₅/ |
| | (GGC)₆ | (GAGG)₅ | (ATCT)₁₄ | (TTTC)₁₅ | (CTTTT)₁₈ | (CTGTG)₉ | (TCCTG)₆ | (CTCTCC)₅ | (CTATAT)₁₁ | (CGT)₅ |
| Alleles | CV106 | CV22 | CV29 | CV37 | CV38 | CV78 | CV104 | CV60 | CV79 | CV94 |
| 1 | 141^{3.60%} | 132^{0.04%} | 85^{9.14%} | 114^{0.16%} | 93^{0.21%} | 138^{0.55%} | 131^{10.63%} | 160^{26.80%} | 113^{0.30%} | 87^{0.06%} |
| 2 | 145^{9.93%} | 137^{0.73%} | 89^{4.23%} | 117^{18.31%} | 98^{0.69%} | 141^{11.98%} | 136^{21.27%} | 166^{14.22%} | 133^{0.30%} | 115^{0.03%} |
| 3 | 148^{13.24%} | 139^{7.74%} | 93^{10.02%} | 118^{0.11%} | 102^{0.90%} | 144^{1.72%} | 141^{8.91%} | 168^{0.60%} | 136^{10.39%} | 135^{0.03%} |
| 4 | 151^{22.30%} | 142^{0.42%} | 97^{9.23%} | 121^{18.31%} | 104^{7.96%} | 147^{15.87%} | 146^{18.50%} | 172^{25.45%} | 138^{2.37%} | 144^{0.03%} |
| 5 | 154^{11.21%} | 144^{1.81%} | 101^{5.23%} | 125^{14.09%} | 109^{6.04%} | 150^{17.83%} | 151^{19.96%} | 178^{15.49%} | 142^{26.71%} | 167^{0.03%} |
| 6 | 156^{5.98%} | 146^{0.12%} | 105^{5.30%} | 129^{10.66%} | 114^{11.23%} | 152^{4.36%} | 156^{20.67%} | 183^{16.62%} | 144^{0.30%} | 170^{0.03%} |
| 7 | 157^{16.95%} | 148^{13.28%} | 109^{6.25%} | 138^{0.05%} | 119^{10.71%} | 155^{1.44%} | 162^{0.04%} | 189^{0.82%} | 148^{19.58%} | 181^{0.03%} |
| 8 | 160^{16.80%} | 150^{0.22%} | 113^{9.29%} | 142^{0.11%} | 124^{3.80%} | 158^{0.81%} | 167^{0.02%} | | 155^{14.54%} | 183^{0.14%} |
| 9 | | 152^{15.48%} | 117^{4.66%} | 146^{2.57%} | 130^{1.05%} | 160^{12.85%} | | | 161^{19.29%} | 186^{28.37%} |
| 10 | | 156^{5.06%} | 121^{5.98%} | 151^{11.55%} | 135^{0.06%} | 163^{1.88%} | | | 163^{0.30%} | 189^{0.06%} |
| 11 | | 158^{1.63%} | 125^{12.73%} | 155^{8.71%} | 140^{4.68%} | 165^{1.44%} | | | 167^{4.15%} | 192^{22.35%} |
| 12 | | 160^{20.42%} | 129^{12.54%} | 159^{5.48%} | 145^{0.32%} | 168^{5.75%} | | | 174^{0.30%} | 198^{25.59%} |
| 13 | | 164^{15.70%} | 133^{3.84%} | 163^{2.64%} | 151^{4.09%} | 170^{4.96%} | | | 179^{0.59%} | 204^{18.27%} |
| 14 | | 166^{17.25%} | 137^{0.99%} | 167^{5.38%} | 156^{12.24%} | 173^{9.29%} | | | 185^{0.89%} | 221^{4.99%} |
| 15 | | 168^{0.10%} | 142^{0.21%} | 171^{1.86%} | 161^{0.50%} | 175^{4.14%} | | | | |
| 16 | | | 147^{0.35%} | | 167^{0.15%} | 178^{5.12%} | | | | |
| 17 | | | 151^{0.01%} | | 171^{0.08%} | 180^{1.85%} | | | | |
| 18 | | | | | 172^{6.79%} | 183^{0.09%} | | | | |
| 19 | | | | | 176^{0.63%} | 186^{7.34%} | | | | |
| 20 | | | | | 181^{13.03%} | 189^{0.11%} | | | | |
| 21 | | | | | 186^{10.73%} | 191^{15.84%} | | | | |
| 22 | | | | | 192^{0.34%} | 196^{0.81%} | | | | |
| 23 | | | | | 199^{0.13%} | 201^{12.48%} | | | | |
| 24 | | | | | 204^{0.04%} | 206^{1.60%} | | | | |
| 25 | | | | | 209^{1.26%} | 212^{0.02%} | | | | |
| 26 | | | | | 214^{0.01%} | | | | | |
| 27 | | | | | 238^{2.41%} | | | | | |
| Total^{b} | 8/0 | 15/0 | 17/0 | 15/2 | 27/4 | 25/2 | 8/0 | 7/0 | 14/0 | 14/0 |
| PIC | 0.85 | 0.85 | 0.92 | 0.89 | 0.92 | 0.92 | 0.82 | 0.79 | 0.80 | 0.77 |
| value^{c} | | | | | | | | | | |

^{a}Motif sequences and number of repeats observed in the cluster consensus sequences. Size of fragments (alleles) considering an extra A added by conventional Taq polymerases. Unstable positions are underlined and refer to alleles with low PCR amplification associated or not with stutters. Allele frequencies are superscripted as percentage numbers.
^{b}Total number of alleles/total number of unstable positions (low amplification alleles).
^{c}PIC values were calculated with numbers of alleles and frequencies observed in the 1,205 accessions fingerprinted.

### Example 5

Capacity of the Fingerprinting System to Discriminate Saccharum Complex Accessions

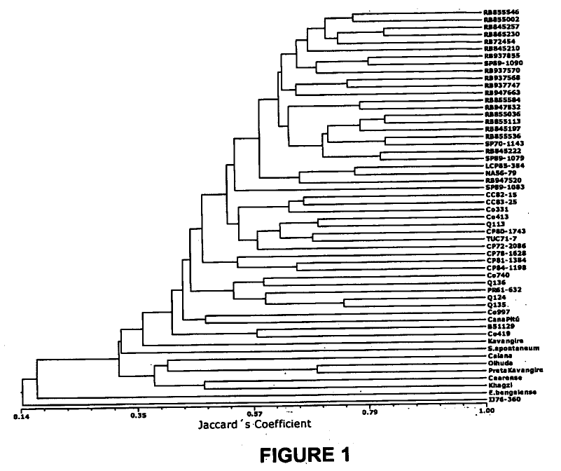

The binary data matrix obtained after genotyping the collection of 1,205 accessions with the ten loci was used to evaluate the discriminatory capacity of the fingerprinting system. Table 10 shows the individual capacities of each locus, all loci combined and the minimum combination of loci that discriminate all accessions with at least two differences (discriminatory alleles). The discriminatory capacities are expressed as the probabilities of genetic identity (Pid, probability of finding identical genotypes for a locus or combination of loci) as well as the percentage of accessions that have a unique genotype for a locus or combination of loci. None of the loci were able to discriminate all accessions individually. All ten loci combined discriminate all accessions with at least three differences. From there, a combination of the smallest number of loci capable of discriminating the collection with at least two differences was searched. Although locus CV78 was capable of discriminating the highest percentage of accessions (60%), it was not chosen to form an optimal combination with other loci due to its PCR (polymerase chain reaction) artifacts (Table 7). Thus, loci CV38 (44%), CV29 (33%), and CV37 (8%) served for this purpose, with the most informative combination reflected by its Pid value of 1 coincident genotype in 10 billions possibilities allied to good scoring characteristics. The discriminatory capacity of this combination is demonstrated in FIG. 1. The dendrogram depicts 54 genotypes belonging to the fingerprinted collection, of which 21 are full-sib commercial and progenitor varieties originated from crosses between varieties RB72454 and SP70-1143 and the rest are genotypes coming from several other foreigner breeding programs and some accessions of related species. In this particular subset of accessions, the minimum number of discriminatory alleles between two individuals was found to be five.

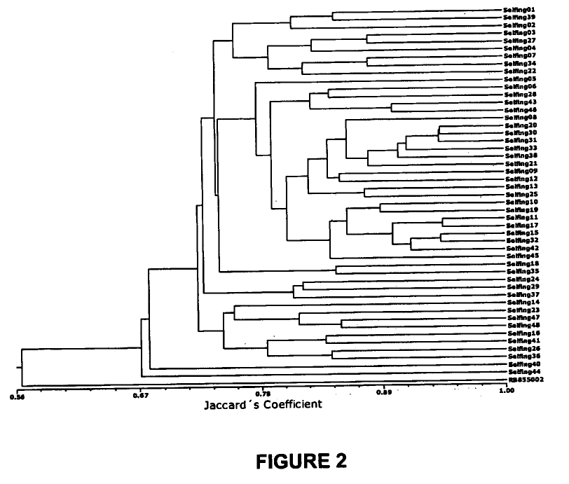

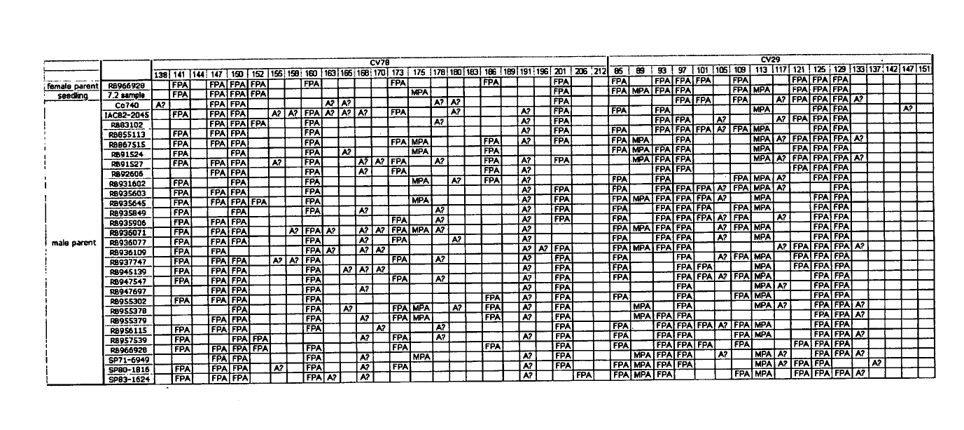

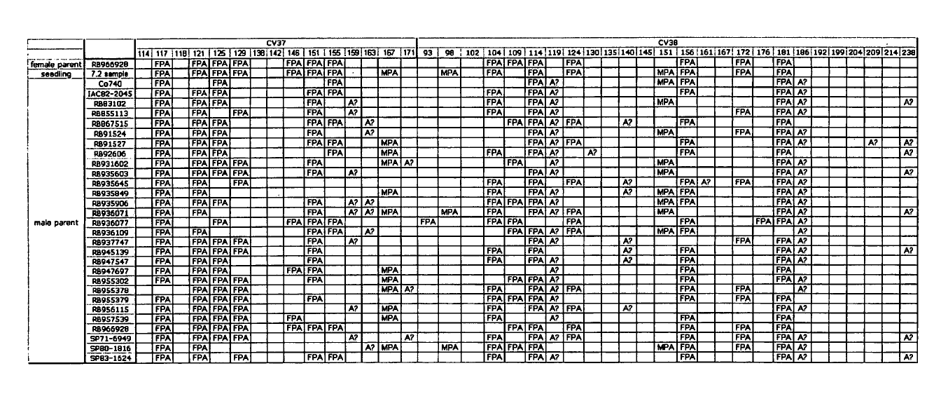

The most extreme case of genetic similarity between two Saccharum complex genotypes in a breeding program is expected to come from progenies of self-pollinated crosses. Thus, the fingerprinting system was evaluated in its capacity to discriminate a population of 48 F1 progenies of a selfing cross of variety RB855002. All possible combinations of loci were tested and in this particular case, a combination of seven loci (CV29, CV37, CV38, CV60, CV79, CV94, and CV106) was necessary to discriminate all individuals with at least two differences (FIG. 2). It is interesting to note that when the optimal combination of three loci was tested (results not shown), the percentage of unique genotypes reached 87.5% (Pid=0.001), with three pairs of genotypes not being discriminated. Addition of locus CV106 increased the unique genotypes to 100% (Pid=0.0005) but with at least one difference. Two differences were only observed with the combination of seven loci described above (Pid=0.00002).

TABLE 10
Discriminatory capacity of the SSR fingerprinting system evaluated in a collection of
1,205 accessions of Saccharum complex and related species sharing diverse levels
of genetic relationship*.

| | Mean number of alleles | | Minimum number | Unique | |
|---|---|---|---|---|---|
| Loci/ | Per | | of discriminatory | genotypes | |
| combination | genotype | Discriminatory | alleles | (%) | P_{id} |
| CV78 | 7.7 | 6.0 | 0 | 60.9 | 0.002 |
| CV38 | 6.9 | 5.1 | 0 | 44.2 | 0.003 |
| CV29 | 7.4 | 4.9 | 0 | 32.7 | 0.003 |
| CV37 | 5.3 | 3.5 | 0 | 7.9 | 0.013 |
| CV22 | 4.9 | 2.8 | 0 | 7.8 | 0.034 |
| CV79 | 3.5 | 2.9 | 0 | 3.2 | 0.062 |
| CV60 | 3.6 | 1.7 | 0 | 2.1 | 0.115 |
| CV106 | 4.5 | 2.9 | 0 | 1.8 | 0.027 |
| CV94 | 3.4 | 1.5 | 0 | 1.5 | 0.213 |
| CV104 | 4.3 | 1.9 | 0 | 1.0 | 0.098 |
| All loci | 51.5 | 38.9 | 3 | 100 | 3.4 × 10⁻¹⁷ |
| combined | | | | | |
| Optimal | 19.6 | 13.5 | 2 | 100 | 1.2 × 10⁻⁷ |
| combination | | | | | |
| (CV29 + CV37 + CV38) | | | | | |

*Low amplification alleles were excluded from this analysis.

### Example 6

Reproducibility of the Fingerprinting System

To evaluate the reproducibility of the fingerprinting system, several experiments were performed to compare DNA sequencer profiles for the ten selected loci (results not shown). These experiments involved the amplification of DNA extracted from the following sample sources: i) different tissues of the same Saccharum spp. genotype (leaf, bud, and root); ii) leaves from the same genotype but originated from several mills located far a part from each other, iii) leaves from the same genotype but from plants in diverse stages of development (age of the plant) and life cycle (plant cane, first ratoon, etc) and iv) in vitro plantlets produced from meristem and calli cultures. In all cases, it was not observed any inconsistency between two high quality profiles generated for the same genotype at any loci fingerprinted. In the largest reproducibility experiment carried out, 783 leaf samples collected from meristem cultured plantlets of 30 commercial varieties were fingerprinted at loci CV29, CV37, and CV38 without any inconsistency being observed among comparable profiles.

### Example 7

Paternity Determination in Polycrosses

Selection of parents generally involves the initial testing of potential candidates where small progenies or families from biparental or polycrosses are evaluated though the regular selection scheme of a particular breeding program. Those pair-wise combinations of male and female that produce elite individuals become proved parents. The same crosses are then made to produce large progenies for the selection of varieties. In the polycrosses, only the mother(s) may be proved at the first stage but several pair-wise combinations must be tested before finding the right father(s). Thus, polycrosses have in theory a much greater chance of revealing proved parents whereas biparental crosses can be made routinely and in large quantities, having higher probability of revealing elite individuals. A polycrossing system where breeders could identify as fast as possible the male parents of selected clones may reduce time and costs of development of new varieties and should have a significant impact in the amount of achieved improvement (sugar, fiber, adaptability, etc).

The fingerprinting system hereof described has been used to identify fathers of promising sugarcane clones under selection in our breeding program and that came from polycrosses involving more than 30 male progenitors. The method used to obtain a categorical answer for establishing paternity was developed for human population studies and forensic applications. The method comprises of: (i) obtaining DNA from the seedling with unknown male parent, female parent and all suppose male parent involved in the cross; (ii) amplifying SSR loci with a set of primers in the samples; (iii) separating amplification products by size (iv) scoring the results of said separation (v) comparing the progeny genotype with the female parent genotype, subtracts the maternal contribution, and compares the remaining paternal gametic contribution with all putative male parent genotypes. The individuals who can not produce the paternal gametic contribution (“paternal alleles”) are excluded, and paternity is assigned to the remaining group (Ellstrand, 1984, Am. Natur. 123: 819-828).

Once the fathers were identified for each clone, biparental crosses involving them and the previously known mothers were carried out and the seeds produced were sown and the resulting seedlings entered the normal selection program. This scheme is repeated every year: the paternity of promising clones evaluated in the first stage of selection are determined with the fingerprinting system, biparental crosses are made in the next crossing season, seeds from these crosses enter the selection scheme of the next year.

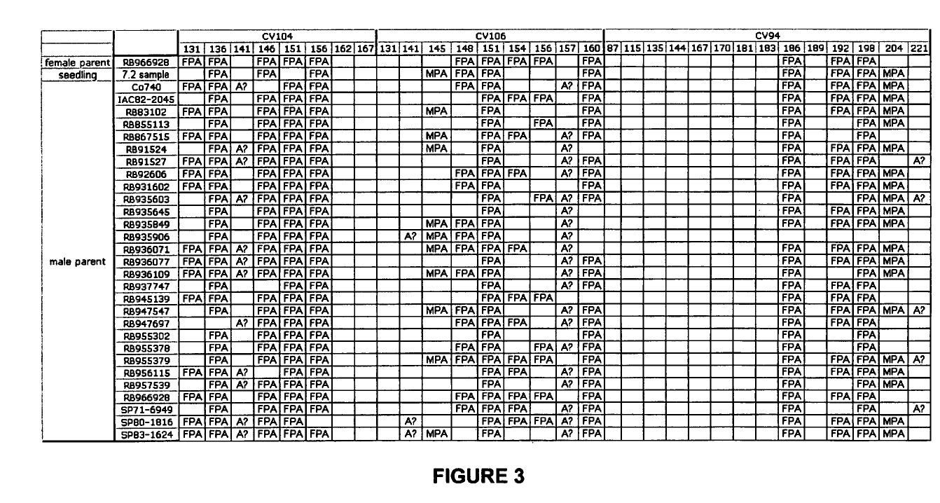

In FIG. 3, RB966928 is the female parent and there are 30 possible male parents, 29 male parents plus the female parent (self-pollination). All samples, seedling, female parent and all male parents were analysed in the fingerprinting system describe herein comparing the progeny genotype with the female parent genotype, subtracts the maternal contribution, and compares the remaining paternal gametic contribution with all putative male parent genotypes. The individuals who cannot produce the paternal gametic contribution (“paternal alleles”) are excluded, and paternity is assigned to the remaining group. The male parent RB936071 is the unique male parent with all MPA, male parent allele (exclusive allele).

We claim:

1. An isolated polynucleotide comprising a sequence selected from the group consisting of:
(i) sequences provided in SEQ ID NO: 1-10; and
(ii) sequences complementary to a sequence provided in SEQ ID NO: 1 -10;
(iii) variants of a sequence of (i) or (ii) having alterations in repeat number into a microsatellite.

2. A primer that binds specifically to an isolated polynucleotide selected from the group consisting of SEQ ID NO: 1-10, wherein the primer comprises at least 6 contiguous nucleotides of a sequence complementary to a sequence provided in SEQ ID NO: 1-10.

3. An isolated primer comprising a sequence selected from the group consisting of:
(i) sequences provided in SEQ ID NO: 11-30;
(ii) sequences complementary to a sequence provided in SEQ ID NO: 11 -30; and
(iii) sequences comprising at least 6 contiguous nucleotides of a sequence provided in SEQ ID NO: 11-30.

4. A method for determining the genotype in a sample from the Saccharum complex comprising the steps of:
(i) obtaining DNA from the sample;
(ii) amplifying simple sequence repeat loci selected from de group consisting of: (i) sequences provided in SEQ ID NO: 1-10; and (ii) sequences complementary to a sequence provided in SEQ ID NO: 1-10; with a set of primers in the DNA selected from the group consisting of (i) sequences provided in SEQ ID NO: 11-30; (ii) sequences complementary to a sequence provided in SEQ ID NO: 11-30, and (iii) sequences comprising at least 6 contiguous nucleotides of a sequence provided in SEQ ID NO: 11-30 to form amplification products of various sizes and labels;
(iii) separating amplification products by size;
(iv) scoring the results of the separation, and
(v) comparing the scored results to results of analysis of DNA from a known species.

5. A method for identifying an accession linking a sample from the Saccharum complex to a plant source, comprising:
(i) determining the identity of DNA in the sample by using the method of claim 4; and
(ii) determining the identity of DNA in a sample from a known plant genotype by using the method of claim 4; and
(iii) comparing the genotypes of both samples to determine the identities of the sample (i).

6. A method for determination of introgression of desirable genomic sequences from samples of Saccharum complex comprising the steps of:
(i) obtaining DNA from the samples;
(ii) amplifying simple sequences repeats loci with a set of primers in DNA selected from the group of primer pairs consisting of SEQ ID NO. 11-30;
(iii) separating amplification products by size;
(iv) identifying male and female parent exclusives alleles, and
(v) identifying offspring individuals with both male and female parent exclusives alleles.

7. A method for selection of progenitors from the Saccharum complex comprising the steps of:
(i) obtaining DNA from the progenitors;
(ii) amplifying simple sequences repeats loci with a set of primers in DNA selected from the group of primer pairs consisting of SEQ ID NO. 11-30 to form amplification products of various sizes and labels;
(iii) separating amplification products by size;
(iv) generating microsatellite binary data with the scored results, and
(v) performing a mathematical treatment of the binary data containing the scored results with the aid of specific genetic similarity estimation softwares for generating parent selection indices.

8. A method for determination of paternity of accessions from the Saccharum complex comprising the steps of:
(i) obtaining DNA from the accessions;
(ii) amplifying simple sequences repeats loci with a set of primers in DNA selected from the group of primer pairs consisting of SEQ ID NO. 11-30 to form amplification products of various sizes and labels;
(iii) separating amplification products by size;
(iv) identifying female and male specific alleles, and
(v) identifying the true male parent.

9. A method for quantification of self-pollination in seedlings samples from the Saccharum complex comprising the steps of:
(i) obtaining DNA from the accessions;
(ii) amplifying simple sequences repeats loci with a set of primers in DNA selected from the group of primer pairs consisting of SEQ ID NO. 11-30 to form amplification products of various sizes and labels;
(iii) separating amplification products by size;
(iv) identifying female and male specific alleles, and
(v) identifying the offspring that contain only female parent exclusives alleles.

10. A kit for detecting accessions from the Saccharum complex, comprising one or more primer pairs suitable for amplifying a nucleotide sequence in a sample from the Saccharum complex.

11. A kit for detecting a polymorphic genetic marker comprising one or more primer pairs suitable for amplifying a nucleotide sequence in a sample from the Saccharum complex.

12. The kit of claim 11, wherein the nucleotide sequence comprises a sequence from the group consisting of: (i) sequences provided in SEQ ID NO: 1-10; (ii) sequences complementary to a sequence provided in SEQ ID NO: 1-10; (iii) sequences provided in SEQ ID NO: 11-30; (iv) sequences complementary to a sequence provided in SEQ ID NO: 11-30, and (v) sequences comprising at least 6 contiguous nucleotides of a sequence provided in SEQ ID NO: 11-30.

13. A method to locate (mapping) genes linked to alleles of quantitative trait loci (QTLs) for the development of MAS (marker-assisted selection) procedures.
